(19) United States
(12) Patent Application Publication
(10) Pub. No.: US 2015/0031014 A1
(43) Pub. Date: Jan. 29, 2015

(54) DETECTING ANALYTES WITH A PH METER

(71) Applicant: The Board of Trustees of the University of Illinois, Urbana, IL (US)

(72) Inventors: Yi Lu, Champaign, IL (US); Yu Xiang, Urbana, IL (US); Zhe Shen, Urbana, IL (US); JingJing Zhang, Champaign, IL (US)

(73) Assignee: The Board of Trustees of the University of Illinois, Urbana, IL (US)

(21) Appl. No.: 14/341,396

(22) Filed: Jul. 25, 2014

Related U.S. Application Data
(60) Provisional application No. 61/858,333, filed on Jul. 25, 2013.

(51) Int. Cl. G01N 33/543; G01N 33/58; G01N 33/68; G01N 33/558; G01N 33/94; G01N 33/84

(52) U.S. Cl. CPC G01N 33/54306; G01N 33/946; G01N 33/84; G01N 33/68; G01N 33/558; G01N 33/581
USPC 435/5; 435/6.11; 435/7.94; 435/287.2

(57) ABSTRACT

Provided herein are sensors, kits that include such sensors, and methods for making and using such sensors. The sensors permit detection of a broad array of target molecules, such as nucleic acids (e.g., DNA and RNA), proteins, toxins, pathogens, cells, and metals, and can be used in combination with pH meters and pH paper. Thus, this disclosure provides a new methodology that allows pH meters and pH paper to be used for the detection of analytes other than pH.

### CROSS-REFERENCE TO RELATED APPLICATION

This application claims priority to U.S. Provisional Application No. 61/858,333 filed Jul. 25, 2013, herein incorporated by reference.

### ACKNOWLEDGMENT OF GOVERNMENT SUPPORT

This invention was made with government support under DE-FG02-08ER64568 awarded by the US Department of Energy and under ES16865 awarded by the National Institutes of Health. The government has certain rights in the invention.

### FIELD

This application relates to sensors, kits that include such sensors, and methods for making and using such sensors. The sensors permit detection of a broad array of target molecules, such as nucleic acids (e.g., DNA and RNA), proteins, toxins, pathogens, cells, and metals, and can be used in combination with pH meters and pH paper.

### BACKGROUND

Developing new methods for quantitative detection of analytes at the point of interest can facilitate on site and real-time monitor of hazardous substances to realize quick response and early treatment.¹ These methods, with low cost and wide accessibility, can also enable the public to do self-detection or self-diagnosis, relieving the burden of environment institutes and medical centers to conduct intense screening tests in different locations between whiles. Most traditional methods of instrument analysis, though very efficient in analyte quantification, require sophisticated device or specific laboratory settings, so that they are generally not suitable for field applications. In contrast, portable devices can be taken along for the detection of analytes at any point of interest.

Despite of the promise, only a few portable devices are successfully commercialized and widely available for public use, such as portable pH meters and personal glucose meters.^{2,3} These portable meters are designed for the detection of only a few limited types of analytes, such as pH and glucose. Therefore, new methods are needed to enable the public to use those portable devices for the detection of more analytes of interest. A general methodology to use DNA-linked invertase to transform the quantitative information of many non-glucose analytes into glucose and thus have achieved their quantification using personal glucose meters.⁴⁻⁶ The present disclosure takes the advantages of portable pH meters to develop a new and general approach to use portable pH meters for the detection of analytes other than pH.

Currently, pH meter is one of the most popular meters used for environment pH monitoring and research. Many types of portable handheld pH meters are commercially available in stores worldwide. They are widely used by the public for home use, such as detecting the pH of drinking water, swimming pool or soil in garden. We envision that, if such a popular public device can be used for the detection of other analytes related to environment and health, it can significantly facilitate the self-detection of many analytes other than pH by the public.

By an enzyme-catalyzed reaction that can induce pH change to the testing solution, we have successfully converted the concentration of an environment pollutant Pb²⁺, a recreational drug cocaine and a protein toxin ricin into pH change through functional DNA recognition and achieved quantitative detection of Pb²⁺, cocaine and ricin by portable pH meters. Because a broad range of analytes such as metal ions, organics, proteins and cells can be recognized by functional DNAs,⁷ the method in this work can serve as a general methodology for the detection of many other analytes using portable pH meters.

### SUMMARY

The present application discloses sensors, and methods of making such sensors, that can be used to detect a target agent, using pH change as a read-out. The disclosure takes advantage of the fact that glucose oxidase (GOx) can convert glucose into gluconic acid, which is an acidifier that can decrease the pH of a solution, which can be detected using a pH meter or pH paper.

Provided herein are methods for detecting a target or a plurality of targets. Such methods include contacting a sample (such as a test sample) with a recognition molecule that is specific for the target of interest, and with a solid support comprising glucose oxidase. The sample is incubated under conditions sufficient to allow the target in the sample to bind to the recognition molecule and to release the glucose oxidase from the solid support. The solid support and the released GOx are separated (or the released GOx is moved to another region of the solid support). The released or moved GOx is contacted with glucose, thereby generating gluconic acid, which can decrease the pH of a solution. The pH is detected, for example using a pH meter or pH paper. In some examples, detection of a significant decrease in pH indicates the presence of the target agent in the sample, and an absence of detected significant decrease in pH indicates the absence of the target agent in the sample. The detection of the target can be qualitative, semi-quantitative, or quantitative. Examples of solid supports include but are not limited to beads (e.g., magnetic beads), graphene oxide, lateral flow devices, or microfluidic devices. Examples of targets that can be detected, include but are not limited to metal ions, microbes, cytokines, hormones, cells, nucleic acid molecules, spores, proteins, recreational drugs, small organic molecules, and toxins.

In one example, the solid support includes a first nucleic acid molecule having a 5′-end and a 3′-end (such as the 5′-end), wherein the first nucleic acid is attached to the solid support by one of the ends, and a second nucleic acid molecule having a 5′-end and a 3′-end. In one example the 5′-end of the second nucleic acid molecule is hybridized to the 3′-end of the first nucleic acid molecule and wherein the 3′-end of the second nucleic acid molecule includes the GOx. In another example the 3′-end of the second nucleic acid molecule is hybridized to the 5′-end of the first nucleic acid molecule and wherein the 5′-end of the second nucleic acid molecule includes the GOx. In some examples, the recognition molecule is a DNAzyme or RNAzyme specific for the target. The DNAzyme or RNAzyme has an enzyme strand, a substrate strand, and in some examples an RNA base in the substrate strand, wherein binding of the target to the DNAzyme or RNAzyme cleaves the substrate strand at the RNA base into a 5′-end piece and a 3′-end piece, wherein the 5′-end piece of the substrate strand is complementary to the first nucleic acid molecule, and wherein the 5′-end piece of the substrate strand displaces the second nucleic acid molecule having the GOx from the first nucleic acid molecule, thereby releasing the GOx from the solid support.

In one example, the solid support includes first nucleic acid molecule having a 5′-end and a 3′-end, wherein the first nucleic acid is attached to the solid support by the one of the ends (such as the 3′-end). The solid support also includes a second nucleic acid molecule having a 5′-end and a 3′-end, wherein the 3′-end of the second nucleic acid molecule is proximal to the 5′-end of the first nucleic acid molecule (and in some example attached or hybridized) and wherein the 5′-end of the second nucleic acid molecule has GOx attached. The solid support also includes a third nucleic acid molecule, an aptamer specific for the target, wherein the aptamer nucleic acid molecule has a 5′-end and a 3′-end, wherein the aptamer nucleic acid molecule is complementary and hybridizes to the first nucleic acid molecule and to the second nucleic acid molecule. In some examples, the 3′-end of the aptamer nucleic acid molecule is not hybridized to the first or second nucleic acid molecule. Binding of the target to the aptamer results in a conformational change in the aptamer nucleic acid molecule and displaces the second nucleic acid molecule having the GOx from the aptamer nucleic acid molecule, thereby releasing the second nucleic acid and the GOx from the solid support.

In one example, the solid support includes an aptamer recognition molecule specific for the target, wherein the aptamer is conjugated with GOx and attached to the solid support by π-π stacking. Binding of the target to the aptamer results in a conformational change in the aptamer and displaces the nucleic acid molecule and its attached GOx from the solid support, thereby releasing the GOx from the solid support.

In one example, the assay is a competitive assay. For example, the recognition molecule can be bound to (a) the solid support and to (b) a target-GOx conjugate, under conditions sufficient to allow the target in the sample to compete with the target-GOx conjugate for binding to the recognition molecule on the solid support and to release the target-GOx conjugate from the solid support. The solid support can be separated from the unbound target and unbound target-GOx conjugate. Either the released target-GOx conjugate or the solid support can be contacted with glucose, under conditions that permit formation of gluconic acid, and the pH measured.

In one example, the assay is a sandwich assay. For example, a first recognition molecule specific for the target is contacted with a sample under conditions sufficient to allow the target in the sample to bind to the first recognition molecule, thereby creating a first recognition molecule-target complex, wherein the first recognition molecule is attached to a solid support. The first recognition molecule-target complex is contacted a second recognition molecule specific for the target conjugated to GOx, thereby creating a first recognition molecule-target-second recognition molecule-GOx complex. This is contacted with glucose, under conditions that permit formation of gluconic acid, and the pH measured.

Also provided are sensors, which can be part of another device, such as a later flow device or a microfluidic device. In one example the sensor includes a solid support that includes a first nucleic acid molecule having a 5′-end and a 3′-end, wherein the first nucleic acid is attached to the solid support by one end (e.g., 5′-end), and wherein the first nucleic acid is complementary to a 5′-end of a substrate strand of a DNAzyme or RNAzyme specific for a target that can be detected by the sensor. The sensor also has a second nucleic acid molecule having a 5′-end and a 3′-end, wherein the 5′-end of the second nucleic acid molecule is hybridized to the 3′-end of the first nucleic acid molecule and wherein the 3′-end of the second nucleic acid molecule has attached or conjugated thereto GOx, or wherein the 3′-end of the second nucleic acid molecule is hybridized to the 5′-end of the first nucleic acid molecule and wherein the 5′-end of the second nucleic acid molecule has attached or conjugated thereto GOx. In one example the sensor includes a solid support that includes a first nucleic acid molecule having a 5′-end and a 3′-end, wherein the first nucleic acid is attached to the solid support by one end; a second nucleic acid molecule having a 5′-end and a 3′-end, wherein the 3′-end of the second nucleic acid molecule is proximal to the 5′-end of the first nucleic acid molecule (and in some example attached or hybridized) and wherein the 5′-end of the second nucleic acid molecule has GOx attached (or vice versa); and an aptamer specific for a target that can be detected by the sensor, wherein the aptamer comprises a nucleic acid molecule having a 5′-end and a 3′-end, wherein the aptamer nucleic acid molecule is complementary and hybridizes to the first nucleic acid molecule and to the second nucleic acid molecule. In some examples the 3′-end of the aptamer nucleic acid molecule is not hybridized to the first or second nucleic acid. In one example the sensor includes a solid support that includes an aptamer nucleic acid molecule conjugated with GOx, wherein the nucleic acid molecule is attached to the solid support by π-π stacking, and wherein the solid support includes graphene oxide. In yet another example the sensor includes a solid support that includes a recognition molecule bound to a target-glucose oxidase complex, wherein in the presence of the target in a sample the amount of target-GOx complex bound to the solid support decreases, and wherein the amount of target in the sample is proportional to the amount of unbound target-GOx complexes.

The disclosure also provides kits that include the disclosed sensors, microfluidic devices, and lateral flow devices. For example, such kits can further include one or more of a buffer, a chart for correlating detected pH and amount of target present, glucose, or glucose oxidase.

Exemplary target agents that can be detected with the disclosed sensors and methods provided herein include a metal, nutritional metal ion (such as calcium, iron, cobalt, magnesium, manganese, molybdenum, zinc, cadmium, or copper), microbe, cytokine, hormone, cell (such as a tumor cell), DNA, RNA, spore (such as an anthrax spore), or toxin. For example, the target agent can be a heavy metal such as mercury (Hg), cadmium (Cd), arsenic (As), chromium (Cr), thallium (Tl), uranium (U), plutonium (Pu), or lead (Pb). In other examples, the target agent is a microbe, such as a virus, bacteria, fungi, or protozoa (such as a microbial antigen or nucleic acid molecule, such as DNA or RNA). In one example the target agent is a spore, such as a bacterial spore, fungal spore or plant spore. For example, Bacillus and Clostridium bacteria (such as C. botulinum, C. perfringens, B. cereus, and B. anthracis) produce spores that can be detected.

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publications with color drawing(s) will be provided by the Office upon request and payment of the necessary fee.

FIG. 1 is a schematic drawing showing the two step strategy to convert the concentration of an analyte (target) in sample into pH change of the testing solution.

FIG. 2 is a graph showing the pH changing process by mixing 50 nM DNA-GOx conjugates with 250 mM glucose in 5 mM pH 7.3 HEPES and 100 mM NaCl.

FIGS. 3A-3D are schematic drawings showing (A) Pb²⁺-induced cleavage of the DNA substrate by the Pb²⁺-dependent 8-17 DNAzyme (SEQ ID NO: 4 substrate strand; SEQ ID NO: 3 enzyme strand); (B) UO₂²⁺-induced cleavage of the DNA substrate by the UO₂²⁺-dependent 39E DNAzyme (SEQ ID NO: 10 substrate strand; SEQ ID NO: 9 enzyme strand). Both reactions yield the cleaved ssDNA product (red) as the invasive DNA; (C) Release of DNA-GOx conjugates from magnetic beads by the invasive DNA (SEQ ID NO: 1 biotin-DNA strand; SEQ ID NO: 2 DNA-GOx strand; nt 1 to 19 of SEQ ID NO: 4, invasive DNA strand, 5′-end of the substrate strand); (D) The invasion and release steps, followed by the conversion of glucose into gluconic acid by the released DNA-GOx conjugates for pH meter measurement.

FIG. 4 is a schematic drawing show a lateral flow device that includes immobilized RNAzyme or DNAzyme and immobilized DNA-GOx conjugate for the detection of a target in a sample using pH as an indicator. Such a device can be used if the recognition molecule is a RNAzyme or DNAzyme.

FIGS. 5A-5C show the modification of aptamers and their immobilization to beads via hybridization with an anchoring nucleic acid (SEQ ID NO: 5 or 11, red) and a DNA-GOx conjugate (SEQ ID NO: 6 or 12, brown). (A) cocaine aptamer (SEQ ID NO: 8), (B) adenosine aptamer (SEQ ID NO: 13), (C) IFN-γ aptamer (SEQ ID NO: 14) on streptavidin-coated metallic beads (MBs) and subsequent release of DNA-GOx conjugates in the presence of these analytes.

FIG. 6 is a schematic drawing showing how magnetic beads that include immobilized DNA-GOx conjugate and aptamer can be used for the detection of a target in a sample using pH as an indicator.

FIG. 7 is a schematic drawing show a lateral flow device that includes immobilized DNA-GOx conjugate for the detection of a target in a sample using pH as an indicator. Such a device can be used if the recognition molecule is an aptamer.

FIG. 8 is a schematic drawing showing a microfluidic flow device that includes immobilized DNA-GOx conjugate for the detection of a target in a sample using pH as an indicator. Such a device can be used if the recognition molecule is an aptamer.

FIG. 9 is a schematic drawing illustrating how ricin can be detected by using portable pH meters (or pH paper) based on graphene oxide. This same principle can be used for any aptamer.

FIGS. 10A and 10B are schematic drawings showing exemplary mechanism of target agent (analyte) detection using pH based on (A) competitive assay and (B) sandwich assay, based on the interaction between first recognition molecule (blue) and second recognition molecule (green) and the target agent (red analyte). pH can be detected with a pH meter or pH paper.

FIGS. 11A and 11B are schematic drawings showing exemplary mechanism of target agent (analyte) detection using pH based on (A) competitive assay and (B) sandwich assay, based on the interaction between first antibody (blue) and second antibody (green) and the target agent (red analyte). pH can be detected with a pH meter or pH paper.

FIGS. 12A and 12B are schematic drawings showing exemplary mechanism of target agent (analyte) detection using pH based on (A) competitive assay and (B) sandwich assay, based on the interaction between first functional nucleic acid (FNA) (blue) and second (FNA (green) and the target agent (red analyte).

FIGS. 13A and 13B are schematic drawings showing exemplary mechanism of target agent (analyte) detection using pH based on (A) competitive assay and (B) sandwich assay, based on the interaction between first nucleic acid (blue) and second nucleic acid (green) and the target agent (red analyte). pH can be detected with a pH meter or pH paper.

FIG. 14 is a schematic drawing showing conjugation of GOx and DNA through Sulfo-SMCC.

FIGS. 15A and 15B are schematic drawings illustrating how Pb²⁺ can be detected in water using portable pH meters (or pH paper) by a DNA invasive approach. (A) shows an overview of the method, and (B) shows details on the competition between the cleaved portion of the substrate strand from the DNZyme can compete with nucleic acid-GOx conjugates immobilized on beads through hybridization. This same principle can be used for any DNAzyme or RNAzyme.

FIGS. 16A and 16B are graphs showing detection of Pb²⁺ in water samples by a portable pH meter. (A) The relationship between measured pH and the concentration of Pb²⁺ in the samples. (B) Selectivity against 80 nM Pb²⁺ (1) over 1 μM Zn²⁺ (2), Cu²⁺ (3), Mg²⁺/Ca²⁺ (4), Cd²⁺ (5) and blank (6).

FIG. 17 is a plot showing quantification of the DNA-GOx conjugates released by samples containing different amounts of Pb²⁺ through fluorescein-labeled DNA-GOx conjugates.

FIG. 18 is a schematic drawing illustrating how cocaine can be detected using portable pH meters (or pH paper). This same principle can be used for any aptamer.

FIG. 19 is a graph showing detection of cocaine in water samples by a portable pH meter. Black squares (bottom): cocaine. Red squares (top): adenosine as control.

FIG. 20 is a graph showing quantification of the DNA-GOx conjugates released by samples containing different amounts of cocaine through fluorescein-labeled DNA-GOx conjugates.

FIGS. 21A-21D. (A) Quantification of the ricin aptamer released by samples containing different amounts of ricin (0-1.4 μg/mL) through fluorescein-labeled DNA. Inset: relationship between fluorescence enhancement and ricin concentration. (B) The relationship between measured pH and the concentration of ricin in 10 mM HEPES buffer by a portable pH meter. (C) Selectivity of ricin detection using pH meter: 100 ng/mL ricin (1) 1 μg/mL BSA (2) 1 μg/mL streptavidin (3) 1 μg/mL aflatoxin (4) and 1 μg/mL biotin (5). (D) Ricin detection in 2% milk using a portable pH meter.

### SEQUENCE LISTING

The nucleic acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. All strands are shown 5′ to 3′ unless otherwise indicated.

SEQ ID NOs: 1 and 2 are sequences used for DNA-GOx immobilization onto beads to detect lead. The 3′-end of SEQ ID NO: 1 hybridizes to the 5′-end of SEQ ID NO: 2.

SEQ ID NO: 3 and 4 are components of a lead-dependent DNAzyme, namely the enzyme strand (SEQ ID NO: 3) and the substrate strand (SEQ ID NO: 4).

SEQ ID NO: 5 is a biotin DNA sequence used for DNA-GOx immobilization onto beads to detect cocaine.

SEQ ID NO: 6 is a sequence for DNA-GOx conjugation for cocaine detection.

SEQ ID NO: 7 is a sequence for DNA-GOx conjugation for ricin detection.

SEQ ID NO: 8 is a sequence of a cocaine aptamer.

SEQ ID NOs: 9 and 10 are components of a lead-dependent DNAzyme, namely the enzyme strand (SEQ ID NO: 9) and the substrate strand (SEQ ID NO: 10).

SEQ ID NO: 11 is a biotin DNA sequence used for DNA-GOx immobilization onto beads to detect IFN-γ.

SEQ ID NO: 12 is a sequence for DNA-GOx conjugation for cocaine detection.

SEQ ID NO: 13 is a sequence of an adenosine aptamer.

SEQ ID NO: 14 is a sequence of an IFN-γ aptamer.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2nd ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

All publications, patent applications, patents, and other references mentioned herein are incorporated by reference in their entirety for all purposes. All sequences associated with the GenBank® Accession numbers mentioned herein are incorporated by reference in their entirety as were present on Jul. 25, 2014, to the extent permissible by applicable rules and/or law.

In order to facilitate review of the various embodiments of the disclosure, the following explanations of specific terms are provided:

3′ end: The end of a nucleic acid molecule that does not have a nucleotide bound to it 3′ of the terminal residue.

5′ end: The end of a nucleic acid sequence where the 5′ position of the terminal residue is not bound by a nucleotide.

Antibody (Ab): Immunoglobulin molecules and immunologically active portions of immunoglobulin molecules, that is, molecules that contain an antigen binding site that specifically binds (immunoreacts with) an antigen (such as a target protein). Exemplary antibodies include monoclonal, polyclonal, camelid, and humanized antibodies, such as those that are specific for a target.

In some examples, an antibody has a high binding affinity for a target, such as a binding affinity of at least about 1×10⁻⁸ M, at least about 1.5×10⁻⁸, at least about 2.0×10⁻⁸, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M. In certain embodiments, an antibody that binds to target has a dissociation constant (Kd) of ≦104 nM, ≦100 nM, ≦10 nM, ≦1 nM, ≦0.1 nM, ≦0.01 nM, or ≦0.001 nM (e.g., 10⁻⁸M or less, e.g., from 10⁻⁸M to 10⁻¹³M, e.g., from 10⁻⁹ M to 10⁻¹³ M). In one embodiment, Kd is measured by a radiolabeled antigen binding assay (RIA) performed with the Fab version of an antibody of interest and its antigen (see, e.g., Chen et al., J. Mol. Biol. 293:865-881, 1999). In another example, Kd is measured using surface plasmon resonance assays using a BIACORES-2000 or a BIACORES-3000 (BIAcore, Inc., Piscataway, N.J.) at 25° C. with immobilized antigen CM5 chips at about 10 response units (RU). Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorptionlionization time-of-flight mass spectrometry, microcytometry, microarray, microscopy, fluorescence activated cell sorting (FACS), and flow cytometry.

A naturally occurring antibody (such as IgG, IgM, IgD) includes four polypeptide chains, two heavy (H) chains and two light (L) chains interconnected by disulfide bonds. As used herein, the term antibody also includes recombinant antibodies produced by expression of a nucleic acid that encodes one or more antibody chains in a cell (for example see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol. 2:239, 1984).

The term antibody also includes an antigen binding fragment of a naturally occurring or recombinant antibody. Specific, non-limiting examples of binding fragments encompassed within the term antibody include Fab, (Fab′)₂, Fv, and single-chain Fv (scFv). Fab is the fragment that contains a monovalent antigen-binding fragment of an antibody molecule produced by digestion of whole antibody with the enzyme papain to yield an intact light chain and a portion of one heavy chain or equivalently by genetic engineering. Fab′ is the fragment of an antibody molecule obtained by treating whole antibody with pepsin, followed by reduction, to yield an intact light chain and a portion of the heavy chain; two Fab′ fragments are obtained per antibody molecule. (Fab′)₂ is the fragment of the antibody obtained by treating whole antibody with the enzyme pepsin without subsequent reduction or equivalently by genetic engineering. F(Ab′)₂ is a dimer of two FAb′ fragments held together by disulfide bonds. Fv is a genetically engineered fragment containing the variable region of the light chain and the variable region of the heavy chain expressed as two chains. Single chain antibody (“SCA”) is a genetically engineered molecule containing the variable region of the light chain, the variable region of the heavy chain, linked by a suitable polypeptide linker as a genetically fused single chain molecule. Methods of making these fragments are routine in the art.

Antigen: A molecule that can stimulate the production of antibodies or a T cell response in an animal, including compositions that are injected or absorbed into an animal. Antigens are usually proteins or polysaccharides. An epitope is an antigenic determinant, that is, particular chemical groups or peptide sequences on a molecule that elicit a specific immune response. An antibody binds a particular antigenic epitope. The binding of an antibody to a particular antigen or epitope of an antigen can be used to determine if a particular antigen (such as a target antigen or antigen of interest) is present in a sample.

Aptamer: Single stranded nucleic acid molecules (such as DNA or RNA) that bind a specific target agent (such as a protein or small organic molecule) with high affinity and specificity (e.g., as high as 10⁻¹⁴ M), and upon binding to the target, the ss nucleic acid molecule undergoes a conformational change and forms a tertiary structure. They are typically around 15 to 60 nt in length, but some are longer (e.g., over 200 nt). Thus, in some examples, aptamers are at least 15 nt, at least 20 nt, at least 25 nt, at least 30 nt, at least 50 nt, at least 60 nt, at least 75 nt, at least 100 nt, at least 150 nt, at least 200 nt, such as 15 to 250 nt, 15 to 200 nt, or 20 to 50 nt.

Aptamers are known in the art and have been obtained through a combinatorial selection process called systematic evolution of ligands by exponential enrichment (SELEX) (see for example Ellington et al., Nature 1990, 346, 818-822; Tuerk and Gold Science 1990, 249, 505-510; Liu et al., Chem. Rev. 2009, 109, 1948-1998; Shamah et al., Acc. Chem. Res. 2008, 41, 130-138; Famulok, et al., Chem. Rev. 2007, 107, 3715-3743; Manimala et al., Recent Dev. Nucleic Acids Res. 2004, 1, 207-231; Famulok et al., Acc. Chem. Res. 2000, 33, 591-599; Hesselberth, et al., Rev. Mol. Biotech. 2000, 74, 15-25; Wilson et al., Annu. Rev. Biochem. 1999, 68, 611-647; Morris et al., Proc. Natl. Acad. Sci. U.S.A. 1998, 95, 2902-2907). In such a process, DNA or RNA molecules that are capable of binding a target molecule of interest are selected from a nucleic acid library consisting of 10¹⁴-10¹⁵ different sequences through iterative steps of selection, amplification and mutation. Aptamers that are specific to a wide range of targets from small organic molecules such as adenosine, to proteins such as thrombin, and even viruses and cells have been identified (Liu et al., Chem. Rev. 2009, 109, 1948-1998; Lee et al., Nucleic Acids Res. 2004, 32, D95-D100; Navani and Li, Curr. Opin. Chem. Biol. 2006, 10, 272-281; Song et al., TrAC, Trends Anal. Chem. 2008, 27, 108-117). The affinity of the aptamers towards their targets can rival that of antibodies, with dissociation constants in as low as the picomolar range (Morris et al., Proc. Natl. Acad. Sci. U.S.A. 1998, 95, 2902-2907; Green et al., Biochemistry 1996, 35, 14413-14424).

Binding: An association between two substances or molecules, such as the hybridization of one nucleic acid molecule to another (or itself), the association of an antibody, aptamer or DNAzyme with a peptide or small organic molecule, the association of a protein with another protein or nucleic acid molecule, or the association between a hapten and an antibody. Binding can be detected by any procedure known to one skilled in the art, for example using the methods provided herein.

One molecule is said to “specifically bind” to another molecule when a particular agent (a “specific binding agent”, such as a recognition molecule) can specifically react with a particular target, for example to specifically immunoreact with a target, or to specifically bind to a particular target. The binding is a non-random binding reaction, for example between a recognition molecule (such as a nucleic acid or antibody) and a target (such as a cell, protein, small organic molecule, metal, DNA or RNA). Binding specificity of can determined from the reference point of the ability of the recognition molecule to differentially bind the specific target and an unrelated molecule, and therefore distinguish between two different molecules. For example, an oligonucleotide molecule binds or stably binds to a target nucleic acid molecule if a sufficient amount of the oligonucleotide molecule forms base pairs or is hybridized to its target nucleic acid molecule, to permit detection of that binding.

In particular examples, two compounds are said to specifically bind when the binding constant for complex formation between the components exceeds about 10⁴ L/mol, for example, exceeds about 10⁶ L/mol, exceeds about 10⁸ L/mol, or exceeds about 10¹⁰ L/mol. The binding constant for two components can be determined using methods that are well known in the art.

Contact: To bring one agent into close proximity to another agent, thereby permitting the agents to interact. For example, a sample can be applied to or mixed with a sensor disclosed herein (such as beads, lateral flow strips or a microfluidic device), thereby permitting detection of target molecules in the sample that are specifically recognized by a recognition molecule (e.g., aptamer, antibody, nucleic acid molecule, or DNAzyme) that is part of the sensor.

Detect: To determine if a particular agent is present or absent, and in some example further includes semi-quantification or quantification of the agent if detected.

Deoxyribozyme (DNAzyme): Functional DNA molecules that display catalytic activity toward a specific target. Also referred to as catalytic DNAs. DNAzymes typically contain a substrate strand (which can include a single RNA base) and an enzyme strand that recognizes a target. DNAzymes show high catalytic hydrolytic cleavage activities toward specific substrates (e.g., targets). In the presence of the specific target, the target will bind to the enzyme strand, resulting in a conformational change in the DNAzyme, and cleavage of the substrate strand (e.g., at the RNA base).

Numerous DNAzymes have been isolated to display high specificity toward various metal ions such as Pb²⁺ (Breaker, and Joyce, Chem. Biol. 1994, 1, 223-9; Li and Lu, J. Am. Chem. Soc. 2000, 122, 10466-7), Cu²⁺ (Carmi et al., Chem. Biol. 1996, 3, 1039-1046; Cuenoud et al., Nature 1995, 375, 611-614), Zn²⁺ (Santoro et al., J. Am. Chem. Soc. 2000, 122, 2433-243; Li et al., Nucleic Acids Res. 2000, 28, 481-488), Co²⁺ (Mei et al., J. Am. Chem. Soc. 2003, 125, 412-420; Bruesehoff et al., Comb. Chem. High Throughput Screening 2002, 5, 327-335), Mn²⁺ (Wang et al., J. Am. Chem. Soc. 2003, 125, 6880-6881), and UO₂²⁺ (Liu et al., Proc. Nat. Acad. Sci. U.S.A. 2007, 104, 2056-2061).

Functional nucleic acids (FNAs): Nucleic acid molecules (such as DNA or RNA molecules) that can be used as enzymes (for catalysis), receptors (for binding to a target), or both. FNAs include ribozyme and DNAzymes (e.g., see Robertson and Joyce, Nature 1990, 344:467; Breaker and Joyce, Chem. Biol. 1994, 1, 223-229), aptamers (e.g., see Tuerk and Gold, Science 1990, 249, 505), aptazymes (e.g., see Breaker, Curr. Opin. Biotechnol. 2002, 13, 31), and aptamers. Additional examples are provided herein and are known in the art.

Glucose Oxidase (GOx): (EC 1.1.3.4) An oxido-reductase that catalyzes the oxidation of glucose into hydrogen peroxide and D-glucono-δ-lactone, which can be hydrolyzed into gluconic acid (an acidifier), for example in water. Nucleic acid and protein sequences for glucose oxidase are publicly available. For example, GENBANK® Accession Nos.: J05242.1; KF741791.1; X56443.1 and NM_{—}001011574.1 disclose exemplary glucose oxidase nucleic acid sequences, and GENBANK® Accession Nos.: AGI04246.1; AHC55209.1; NP_{—}001011574.1; AAA32695.1 (such as aa 23-605 of this sequence) and AAF59929.2 disclose exemplary glucose oxidase protein sequences, all of which are incorporated by reference as provided by GENBANK® on Jul. 25, 2014. In one example, a glucose oxidase is one from Aspergillus niger. In certain examples, glucose oxidase has at least 80% sequence identity, for example at least 85%, 90%, 95%, or 98% sequence identity to a publicly available glucose oxidase sequence (such as one of the GenBank Accession Nos. above), and is a glucose oxidase which can catalyze the oxidation of glucose into hydrogen peroxide and D-glucono-δ-lactone, which can be hydrolyzed into gluconic acid, for example in water.

Hybridization: Hybridization of a nucleic acid occurs when two nucleic acid molecules undergo an amount of hydrogen bonding to each other. The stringency of hybridization can vary according to the environmental conditions surrounding the nucleic acids, the nature of the hybridization method, and the composition and length of the nucleic acids used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of nucleic acid is hybridized to its complementary strand.

Immobilized: Bound or attached to a surface, such as a solid support. Such attachment can be covalent or non-covalent. In one embodiment, the solid surface is in the form of a bead, a lateral flow strip (or portion thereof, such as a membrane), or a microfluidic device. In some examples, the solid surface can include immobilized recognition molecules that can specifically bind to a target agent, DNA-glucose oxidase molecules, glucose, or combinations thereof. Methods of immobilizing agents to solid supports are known in the art. For example, methods of immobilizing peptides on a solid surface can be found in WO 94/29436, and U.S. Pat. No. 5,858,358. In some examples, agents are immobilized to a support by simply applying the agent in solution to the support, and allowing the solution to dry, thereby immobilizing the agent to the support. In other examples, agents are immobilized to a support using a reactive group, such as an amine, or linkers, such as streptavidin/biotin, thereby immobilizing the agent to the support.

Lateral flow device: An analytical device in the form of a test strip used in lateral flow chromatography, in which a sample fluid, such as one suspected of containing a target, flows (for example by capillary action) through the strip (which is frequently made of bibulous materials such as paper, nitrocellulose, and cellulose). The test sample and any suspended analyte (including target agents) can flow along the strip to a detection zone in which the target agent (if present) interacts with a recognition molecule of the sensors provided herein to indicate a presence, absence and/or quantity of the target agent.

Numerous lateral flow analytical devices are known, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,368,876; 7,799,554; EP 0810436; and WO 92/12428; WO 94/01775; WO 95/16207; and WO 97/06439, each of which is incorporated by reference. Thus, these known later flow devices can be modified using the teachings herein.

pH Meter: Refers to any electronic device for determining the pH of a substance, such as a liquid or semi-solid substance. A typical pH meter includes a measuring probe (e.g., glass electrode) connected to an electronic meter that measures and displays the pH reading. pH meters include any commercially available pH meter, such as a portable pH meter. The disclosure is not limited to a particular brand or source of pH meter, though examples include those available from Omega (Stamford, Conn.), Hanna Instruments (Woonsocket, R.I.), The Lab Depot, Inc. (Dawsonville, Ga.) and Thermo Scientific.

pH Paper: Refers to any filter paper that has been treated with a natural water-soluble dye for determining the pH of a substance, such as a liquid or semi-solid substance. Two types of pH paper are commonly used: litmus paper and universal (Alkacid) paper. The disclosure is not limited to a particular brand or source of pH paper, though examples include those available from Micro Essential Laboratory (B'KLYN, N.Y.)

Recognition molecule: An agent, such as a nucleic acid molecule (including functional nucleic acid molecules), protein, peptide nucleic acid, polymer, small organic molecule, or antibody (or fragment thereof)) that can bind to a target agent with high specificity. Thus, a recognition molecule binds substantially or preferentially only to a defined target. For example a recognition molecule specific for one metal does not bind significantly to other metals. Similarly, a recognition molecule specific for one protein does not bind significantly to other proteins. DIXDC1 polypeptide. The determination that a particular recognition molecule binds substantially only to a target may readily be made by using or adapting routine procedures.

Sensor: A device that responds to physical or chemical stimuli, and produces a detectable signal (directly or indirectly). Thus, sensors can be used to determine whether a target agent is present or absent. In one example, the disclosed sensors include one or more of a recognition molecule that is specific for the target agent, attached to a solid support, glucose oxidase (for example attached to a nucleic acid molecule), and glucose.

Sequence identity: The similarity between amino acid (or nucleotide) sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a protein or nucleic acid can possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of nucleic acid molecule, protein, and coding sequences known in the art and disclosed herein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Target (or target agent): Any substance whose detection is desired, including, but not limited to, a chemical compound, metal (such as a heavy metal), pathogen, toxin, nucleic acid (such as DNA or RNA), or protein (such as a cytokine, hormone or antigen), as well as particular cells (such as a cancer cell or bacterial cell), viruses, or spores.

Under conditions sufficient for: A phrase that is used to describe any environment that permits the desired activity. An example includes contacting an antibody or a nucleic acid probe with a biological sample sufficient to allow detection of one or more target proteins or nucleic acid molecules (e.g., DIXDC1), respectively, in the sample.

### OVERVIEW

This disclosure provides a new methodology that allows pH meters and pH paper to be used for the detection of analytes other than pH, such an environment pollutant (e.g., Pb²⁺), a recreational drug (e.g., cocaine), or a protein toxin (e.g., ricin). Detection limits of 20 nM, 10 μM, and 56 ng/mL were obtained for lead, cocaine, and ricin, respectively. The nanomolar concentration detection limit for lead (lower than EPA regulated level in drinking waters), micromolar concentration detection limit for cocaine, and ng/mL concentration detection limit for ricin were achieved with good selectivity over other similar metal ions, organic compounds and proteins as controls.

The non-pH analytes were recognized by functional DNAs, such as DNAzymes (catalytic cleaving of substrate by DNAzyme specifically in the presence of lead) and aptamers (DNA aptamer structure switching specifically in the presence of cocaine and ricin) and the interaction between the analyte and the corresponding functional DNA caused the release of DNA conjugated to glucose oxidase (DNA-GOx conjugates) from the surface of a solid support into solution. Subsequent removal of the solid support resulted in the solution containing the released DNA-GOx, which further catalyzed the production of gluconic acid from glucose and decreased the pH of the testing solution. Thus, the specific interaction between the target and the functional nucleic acid was converted to a pH signal through oxidation of glucose catalyzed by DNA-GOx conjugates to produce gluconic acid that changes the pH of testing solutions for pH measurement.

Based on these observations, provided herein are methods, sensors, and kits that allow the pH measured by pH meters (such as a portable meter) or pH paper for the detection of one or more target analytes in samples. In this method, the enzymatic reaction not only yielded products that lowered pH, but also realized signal amplification so that nM and μM level of analytes could trigger the pH change of solutions containing mM level of buffer components. Because many other analytes can be recognized by known functional DNAs, the methodology is generally applicable to a broad range of targets. In addition, nucleic acid hybridization assays for DNA and RNA, as well as immunoassays for various targets using antibodies are applicable by detecting changes in pH using similar methods, such as sandwich assays and competitive assays.

Thus, the disclosure provides a completely new methodology to detect various analytes or targets of interest by using commercially available pH meters and pH paper, which are cheap and can be easily accessed by the public. Currently, pH meters and pH paper can only be used to detect the pH of a solution or wet solids such as soils. This disclosure provides a new technology and makes many other substances detectable using pH meters and pH paper. This facile and cost-effective method permits on-site, real time monitoring of analytes related to environment and health, such as Pb²⁺ and cocaine, using a commercially available portable pH meters and pH paper.

### Methods of Detecting Target Agents Using pH as an Indicator

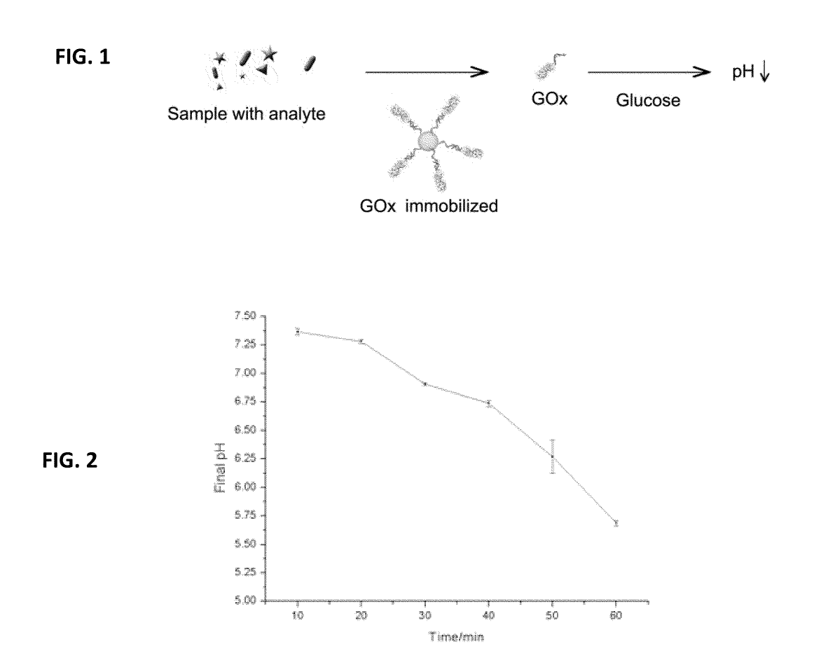

The present disclosure provides methods of detecting a target agent as indicated by a change (e.g., decrease) in pH. Thus, the methods permit for a determination as to whether a target is present in a sample, such as a biological, environmental, or food sample. In some examples, more than one target is detected simultaneously or contemporaneously, such as at least 2, at least 3, at least 4, at least 5, or at least 10 different targets. The inventors have developed a way to use pH meters (such as a portable pH meter) or pH paper to quantify analytes other than pH, by breaking the buffer capacity of mM buffers in a manner dependent on nanomolar (nM) or micromolar (μM) level analytes. To do this, recognition molecules (such as functional DNA) was used to recognize different targets, and then the recognition of low concentrations (nM or μM level) of analytes was converted into a detectable pH change based on glucose oxidase (GOx). GOx is an enzyme capable of catalyzing glucose oxidation to yield acids to change the pH of solution. This is outlined in FIG. 1, which shows that in the presence of a specific analyte or target, if the recognition molecule is a functional DNA (e.g., aptamer or DNAzyme), upon binding of the functional DNA to the target, this induces a structure change of DNA duplex containing DNA-GOx conjugates on a solid support, causing the release of DNA-GOx conjugates from solid support into solution. The released DNA-GOx conjugates then catalyze the oxidation of glucose to change the solution pH. Because the change of pH, the amount of released DNA-GOx conjugate and the concentration of the target are dependent, the quantification of the target can be achieved by the signal of pH change in portable pH meters or pH paper. In some examples, the solution pH is maintained during the target recognition step and then changed in the signal transformation step, so the performance of recognition molecule (e.g., functional DNA or Ab) is not significantly affected.

To confirm the above assumption of changing the pH of buffers by GOx-catalyzed oxidation, the ability of DNA-GOx conjugates to change the pH of a buffer solution by catalyzing the oxidation reaction of glucose was tested. As shown in FIG. 2 a solution containing 50 nM DNA-GOx conjugates and 250 mM glucose in 5 mM HEPES was continuously monitored by a portable pH meter over 1 hour. A time-dependent pH decrease was observed due to the enzymatic reaction yielding gluconic acid.

Thus provided herein are methods for detecting one or more targets. Such methods can include contacting a sample with a recognition molecule specific for the target and a solid support that includes glucose oxidase (GOx), under conditions sufficient to allow the target in the sample to bind to the recognition molecule and to release the glucose oxidase from the solid support (or from a region of the solid support). The released glucose oxidase can be separated from solid support or moved (e.g., flow) to a different area of the solid support (e.g., a different region of a lateral flow strip or microfluidic device). The released GOx is contacted with glucose under conditions that permit the formation of gluconic acid. A change in pH is detected, for example with a pH meter or pH paper. Gluconic acid is an acidifier, and thus will decrease pH if the target is present. Thus, detection of a significant decrease in pH indicates the presence of the target agent in the sample, and an absence of detected significant decrease in pH indicates the absence of the target agent in the sample. In some examples, a significant change is a decrease in pH of at least three times over that from the background, such as at least four times at least five times, at least six times, at least seven times, at least eight times, at least nine time, or at least 10-times over that from the background. In some examples the amount of target is quantified, such as semi-quantified, as the detected pH correlates to an amount of target agent present.

In some examples, the methods include determining or measuring the pH of the solution prior to contacting it with the recognition molecule or GOx, to get a baseline pH reading, from which it can be determined if the pH is altered (e.g., decreased) following the reaction provided with the methods herein.

Targets that can be detected with the disclosed methods include, but are not limited to, a metal, microbe, cytokine, hormone, cell, nucleic acid molecule, spore, protein, small organic molecule, recreational drug, or toxin. The disclosed sensors, which can be part of lateral flow devices or microfluidic devices, can be used in methods for detecting a target agent, for example to diagnose a disease or infection, or to detect exposure to, or the presence of, a particular metal or drug.

In some examples, the methods use a lateral flow device to convert the target into gluconic acid, which can be used to decrease the pH of a solution. In some examples the lateral flow device includes a wicking pad, one or more conjugation pads, one or more membranes, and absorption pad. The sample containing or suspected of containing one or more target agents is applied to the wicking pad. If desired, liquid can be added to the sample, or the sample can be concentrated, before applying it to the wicking pad. The wicking pad ensures a controllable (unilateral) flow of the sample. The sample migrates from one end the lateral flow device to the other because of capillary force. When the target agent in the sample reaches one or more conjugation pads, the target binds to the recognition molecule on the conjugation pad, and releases GOx to the mobile phase, which can travel to a membrane containing glucose. Then, GOx can catalyze the conversion of glucose into gluconic acid, which moves with the flow reaches the absorption pad, wherein the pH is detected (e.g., using pH paper, which may be part of the device, or using a pH meter). The pH detected by a pH meter or pH paper, the GOx released, and target are proportional to each other. This permits quantification of the target agent by determining or measuring the pH after the reaction of the released GOx with glucose. Because of high selectivity of the recognition molecule for its target, interference by other components in the sample is minimal.

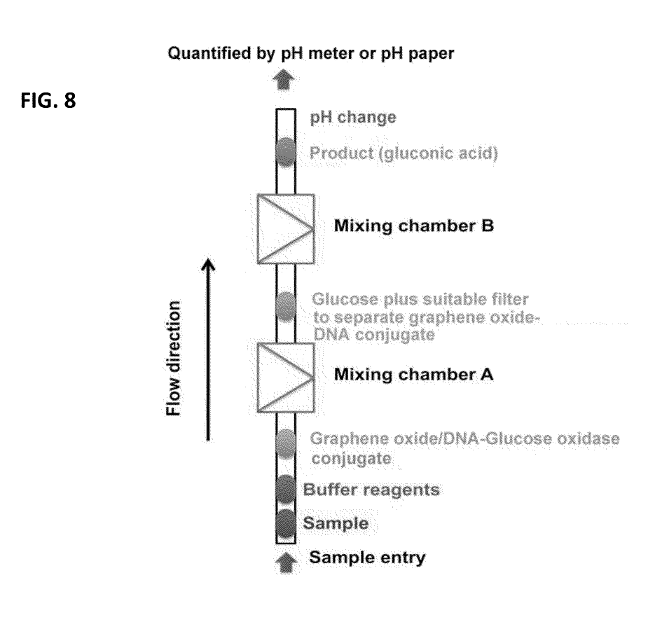

In some examples, the methods use a microfluidic flow device to convert the target into gluconic acid, which can be used to decrease the pH of a solution. The microfluidic device controls the movement of the sample and other liquids, dispenses reagents, and merges or splits a micro-size droplet in the microfluidic device via the voltage applied to the flow versus the device. The test sample is introduced into the microfluidic device and mixed with droplets of buffer reagents (such as red blood cell lysis buffers and suitable buffers for the enzymatic reaction) and starting products. In an example the one or more starting products include the recognition molecule, and GOx, such as a GOx conjugate. The mixture droplet moves into a first mixing chamber for sufficient time to ensure that the recognition molecule can bind the target, and GOx can be released. The released GOx can travel through a filter to remove undesired reagents, and to react with glucose in mixing chamber B to produce gluconic acid. After the completion of the enzymatic reaction (e.g., production of gluconic acid from GOx and glucose) the solution containing gluconic acid moves to the end. Finally, the droplet containing gluconic acid is tested by a pH meter or pH paper after it is released from the microfluidic device. In some examples, the gluconic acid is mixed with a solution prior and the pH of the solution determined. Thus, as shown in FIG. 8, the filter can be designed to separate solid support (e.g., graphene oxide)-GOx-DNA conjugate. In mixing chamber A, the targets react with graphene oxide-GOx-DNA conjugate, and will cause the release of GOx-DNA conjugate from the surface of graphene oxide. When the resulting solution moves to the filter, only the released GOx-DNA conjugate could pass through the filter, and moves into Mixing Chamber B. Thus, glucose can be included t after the filter. The GOx-DNA conjugate will react with glucose in the Mixing Chamber B to produce gluconic acid.

Example when Recognition Molecule is a DNAzyme or RNAzyme

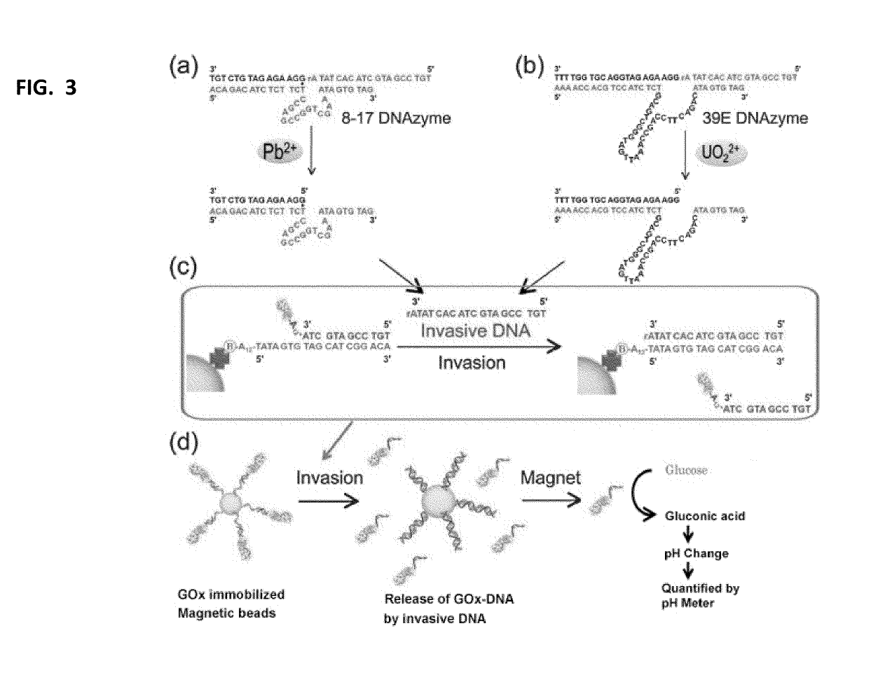

In one example, the solid support used in the method includes a nucleic acid-GOx conjugate (e.g., DNA-GOx) attached thereto. For example, the first nucleic acid of the nucleic acid-GOx conjugate can have a 5′-end and a 3′-end, wherein the first nucleic acid is attached to the solid support, for example by the 5′-end (see FIG. 3C, bottom strand is the first nucleic acid molecule). The second nucleic acid molecule also has a 5′-end and a 3′-end, wherein the 5′-end of the second nucleic acid molecule is hybridized to the 3′-end of the first nucleic acid molecule and wherein the 3′-end of the second nucleic acid molecule has attached thereto GOx (see FIG. 3C, top green strand is the second nucleic acid molecule). For example, the GOx can be attached to the 3′-end of the second nucleic acid via a linker, such as a linker of at least 6 nucleotides (nt), such as at least 7 nt, at least 8 nt, at least 9 nt, at least 10 nt, at least 11 nt, at least 12 nt, at least 13 nt, at least 14 nt, or at least 15 nt, such as 6 nt-20 nt, 6 nt-15 nt, 6 nt-12 nt, 9 nt-12 nt, for example 12 nt. In one example, the linker is a plurality of “As”. One skilled in the art will appreciate that this can be reversed, such that the first nucleic acid molecule can be attached to the solid support by its 3′-end, and the second nucleic acid molecule is hybridized to the 5′-end of the first nucleic acid molecule and wherein the 5′-end of the second nucleic acid molecule has attached thereto GOx.

In some examples, the recognition molecule includes an RNAzyme or DNAzyme specific for the target. As shown in FIGS. 3A and 3B, such recognition molecules can have an enzyme strand (bottom green stand), a substrate strand (top black and red strand), and an optional RNA base (rA) in the substrate strand. Binding of the target to the RNAzyme or DNAzyme cleaves the substrate strand (e.g., at the RNA base) into a 5′-end piece (red, FIGS. 3A, 3B, 3C) and a 3′-end piece (black, FIGS. 3A, 3B). Known RNAzymes and DNAzymes can be used, and modified for the methods and sensors provided herein.

For example, FIGS. 3A and 3B show how a lead and a UO₂²⁺ DNAzyme can be altered to be used with the disclosed methods and sensors. For example, the 5′-end of the substrate strand can be extended such that the entire cleaved 5′-end piece is complementary to the first nucleic acid molecule of the nucleic acid-GOx conjugate attached to the solid support. For example, before the modification, the cleaved 5′-end piece is typically 7 to 10 nt (e.g., 9 nt). However, this can be extended by at least 5 nt, at least 6 nt, at least 7 nt, at least 8 nt, at least 9 nt, at least 10 nt, at least 11 nt, or at least 12 nt, such as 6 nt-9 nt, 9 nt-12 nt, such as 9 nt, wherein the resulting cleaved sequence is designed to be complementary to the first nucleic acid strand attached to the solid support. Because the released oligonucleotide from the cleaved substrate has more matched base pairs with the first anchor DNA than the second DNA attached to the GOx, the former can serve as an invasive DNA to compete with the DNA-GOx conjugates in hybridizing with the biotinylated DNA on the solid support, and induce the release of the DNA-GOx conjugates. In some examples, this 5′-end piece of the substrate strand is complementary to the first nucleic acid molecule of the nucleic acid-GOx conjugate attached to the solid support (such as at least 90%, at least 92%, at least 95%, at least 98% or at least 100% complementarity). In addition, this 5′-end piece of the substrate strand is longer than the DNA-GOx strand, such as at least 5 nt, at least 6 nt, at least 7 nt, at least 8 nt, at least 9 nt, at least 10 nt, at least 11 nt, or at least 12 nt, such as 5 nt-10 nt, 5 nt-12 nt, 6 nt-8 nt, such as 7 nt, longer, and thus will more effectively hybridize to the first nucleic acid molecule than does the nucleic acid-GOx conjugate. As a result, this 5′-end piece of the substrate strand displaces the second nucleic acid molecule comprising the glucose oxidase (which was hybridized to the first nucleic acid molecule of the nucleic acid-GOx conjugate) from the first nucleic acid molecule, thereby releasing the GOx from the solid support (or allowing the GOx to move to a different region of the solid support) (FIGS. 3C and 3D). This released or moved GOx can be contacted with glucose to produce gluconic acid (FIG. 3D).

In some examples, the solid support is a bead. As shown in FIG. 3D, after the GOx is released from the beads into a solution, the beads and the solution containing the GOx can be separated, for example by centrifugation, or by using a magnet if the beads are magnetic. The resulting solution can be contacted with glucose (e.g., glucose added) to allow the formation of gluconic acid, and the pH of the solution determined.

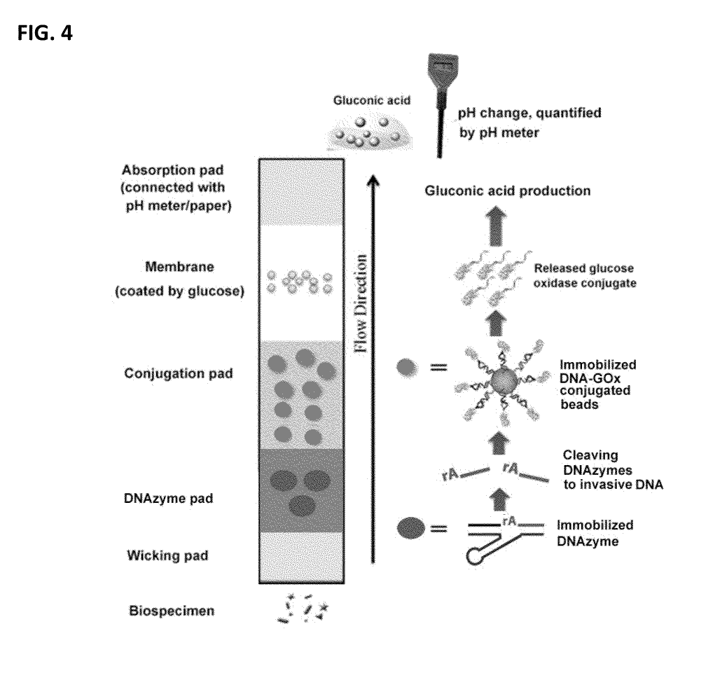

In some examples, the solid support is a lateral flow device (e.g., see FIG. 4). In such examples, the sample can be applied to the device, for example at a wicking pad, and the sample allowed to travel or flow through the device. One region of the lateral flow device includes the RNAzyme or DNAzyme specific for the target, for example on a first conjugation pad (shown in FIG. 4 as DNAzyme pad). The target in the sample (if present) flows through the lateral flow device and binds to the recognition molecule on the lateral flow device, thereby forming a target-recognition molecule (e.g., target-DNAzyme) complex. After formation of the target-recognition molecule complex, this will result in cleavage of the substrate strand of the RNAzyme or DNAzyme. The resulting 5′-end piece of the substrate strand of the RNAzyme or DNAzyme, which is designed as complementary to the nucleic acid strand that hybridizes to the nucleic acid-GOx conjugate attached to the lateral flow device, is allowed to flow to a region of the lateral flow device (e.g., second conjugation pad) containing the nucleic acid-GOx conjugate (e.g., DNA-GOx conjugate). In some examples, the nucleic acid-GOx complex is attached to beads, which are immobilized on another (e.g., second) conjugation pad. Upon reaching the region of the lateral flow strip containing the nucleic acid-GOx conjugate, the 5′-end piece of the substrate strand competes with the nucleic acid-GOx conjugate in hybridizing with the biotinylated DNA on the solid support, and induces the release of the nucleic acid-GOx conjugates (e.g., DNA-GOx conjugates). In this example, instead of separating the solid support from the released GOx, the released GOx is allowed to flow to a different part of the lateral flow device, such as a membrane that includes glucose under conditions that permit the formation of gluconic acid. The resulting gluconic acid can change the pH of a solution, which can be detected with a pH meter or pH paper. In some examples, the lateral flow strip includes pH paper (for example the absorption pad can be pH paper), which can be used as a read-out of pH. In some examples, the resulting droplet is read by a pH meter.

A specific exemplary lateral flow device is shown in FIG. 4. The lateral flow device includes a bibulous lateral flow strip, which can be present in housing material (such as plastic or other material). The lateral flow strip is divided into a proximal wicking pad, a first conjugation pad (containing an immobilized DNAzyme or RNAzyme specific for the target), a second conjugation pad (containing immobilized nucleic acid-GOx, such as DNA-GOx conjugate, which may be present on beads as shown), a membrane coated with glucose, and a distal absorption pad (which can be connected with pH paper or a pH meter). The flow path along strip passes from proximal wicking pad, through the conjugation pads, into the membrane coated with glucose, for eventual collection in absorption pad.

In operation of the particular embodiment of a lateral flow device illustrated in FIG. 4, a fluid sample containing a target of interest (or suspected of containing such), such as a metal target agent, is applied to the wicking pad, for example dropwise or by dipping the end of the device into the sample. If the sample is whole blood, an optional developer fluid can be added to the blood sample to cause hemolysis of the red blood cells and, in some cases, to make an appropriate dilution of the whole blood sample. From the wicking pad, the sample passes, for instance by capillary action, to the first conjugation pad. In the conjugation pad, the target of interest binds the immobilized DNAzyme or RNAzyme. For example, if the DNAzyme or RNAzyme is specific for lead, lead in the sample will bind to the immobilized DNAzyme or RNAzyme contained in the conjugation pad. After this binding, a target-recognition molecule (e.g., target-DNAzyme) complex is formed, resulting in cleavage of the substrate strand of the RNAzyme or DNAzyme. The resulting 5′-end piece of the substrate strand of the RNAzyme or DNAzyme, which is designed as complementary to the nucleic acid strand that hybridize with the DNA-GOx conjugate attached to the second conjugation pad on lateral flow device, is allowed to flow to the second conjugation pad of the lateral flow device containing the nucleic acid-GOx conjugate. Upon reaching the second conjugation pad containing the nucleic acid-GOx conjugate, the 5′-end piece of the substrate strand competes with the DNA-GOx conjugates in hybridizing with the biotinylated DNA on the solid support, and induces the release of the DNA-GOx conjugates. This is shown as “release glucose oxidase conjugate”. This released GOx can subsequently flow to the membrane where the GOx can interact with glucose present on the membrane, thereby producing gluconic acid. The resulting gluconic acid can subsequently flow to the absorption pad, which can be read by a pH meter or contacted with pH paper, wherein detection of a decrease in the pH indicates the presence of target agent in the sample tested.

In some examples, the solid support is a microfluidic device. In examples where the recognition molecule is a DNAzyme or RNAzyme, the test sample is introduced into the microfluidic device and mixed with droplets of buffer reagents (such as red blood cell lysis buffers and suitable buffers for the enzymatic reaction) and starting products. In an example the one or more starting products includes the DNAzyme or RNAzyme. The mixture droplet moves into a first mixing chamber for sufficient time to ensure that the DNAzyme or RNAzyme can bind the target, and that the DNAzyme or RNAzyme is cleaved, producing the 5′-end of the substrate strand. The resulting 5′-end of the substrate strand can be released from the first mixing chamber, and if desired, can travel through a filter to remove undesired reagents. The 5′-end of the substrate strand is allowed to interact with an appropriate nucleic acid-GOx complex (such as one attached to beads), for example in a second mixing chamber, under conditions that allow the GOx to be released. The released GOx can be released from the second mixing chamber, and allowed to react with glucose to form gluconic acid, for example in a third mixing chamber, under conditions that allow for completion of the enzymatic reaction and the gluconic acid to be released. Finally, the droplet containing gluconic acid is tested by a pH meter or pH paper after it is released from the microfluidic device. In some examples, the gluconic acid is mixed with a solution prior and the pH of the solution determined.

Example when Recognition Molecule is an Aptamer

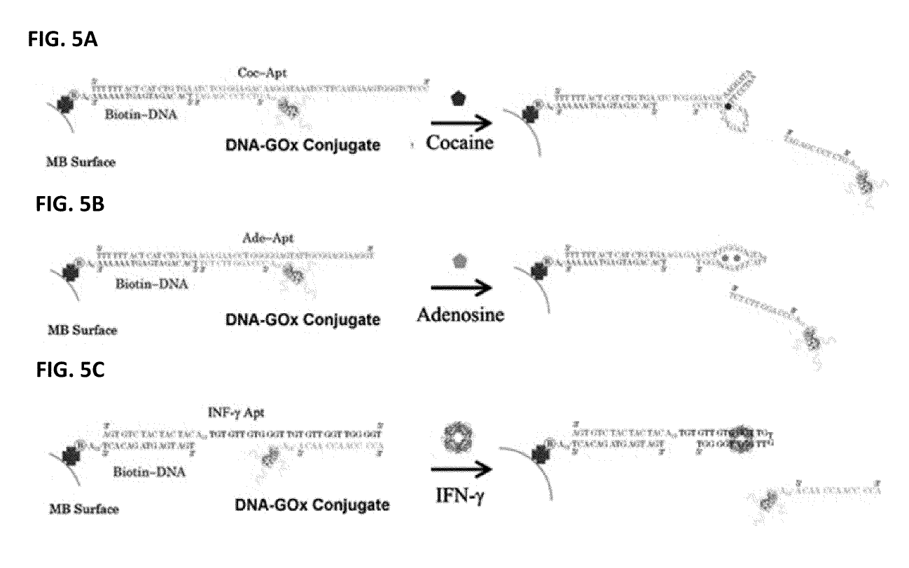

In one example, the solid support used in the method includes an anchoring nucleic acid molecule, a nucleic acid-GOx conjugate (e.g., DNA-GOx), as well as an aptamer recognition molecule specific for the target (see FIGS. 5A-5C). In one example, three different nucleic acid molecules are used, wherein one is attached directly to the solid support (the anchoring or capture nucleic acid molecule), and the other two nucleic acid molecules are attached to the solid support via hybridization (the aptamer hybridizes to both the anchoring nucleic acid and the nucleic acid-GOx conjugate). For example, a DNA sandwich structure can be assembled on a solid support by connecting a nucleic acid-GOx conjugate (e.g., DNA-GOx conjugate) to the capture nucleic acid (e.g., biotin-DNA) through simultaneous hybridization with the aptamer. The target-specific structure switching of the aptamer in the presence of target causes the disassembly of the DNA sandwich structure.

For example, the first nucleic acid can have a 5′-end and a 3′-end, wherein the first nucleic acid is attached to the solid support, for example by the 3′-end (FIGS. 5A, 5B) or the 5′-end (FIG. 5C). In some examples, the first nucleic acid is labeled with biotin (e.g., at its 3′-end), and thus can be attached to streptavidin-coated solid supports. The second nucleic acid molecule also has a 5′-end and a 3′-end, which includes a GOx one end (e.g., 5′-end see FIGS. 5A and 5B, 3′-end see FIG. 5C). The other end of the second nucleic acid molecule is proximal (e.g., attached or hybridized) to the 5′-end of the first nucleic acid molecule (FIG. 5A, 5B) if the 3′-end of first nucleic acid is attached to the solid support, or hybridizes to the 5′-end of the aptamer (FIG. 5C). For example, the GOx can be attached to one end of the second nucleic acid via a linker, such as a linker of at least 6 nucleotides (nt), such as at least 7 nt, at least 8 nt, at least 9 nt, at least 10 nt, at least 11 nt, at least 12 nt, at least 13 nt, at least 14 nt, or at least 15 nt, such as 6 nt-20 nt, 6 nt-15 nt, 6 nt-12 nt, 9 nt-12 nt, for example 12 nt. In one example, the linker is a plurality of “A”s. The third nucleic acid molecule is the aptamer specific for the target, which has a 5′-end and a 3′-end. In one example (FIGS. 5A and 5B), the 5′-end of the aptamer is complementary and hybridizes to the first nucleic acid molecule, and the second nucleic acid molecule is complementary and hybridizes to the middle of the aptamer. In some examples, the 3′-end of the aptamer nucleic acid molecule is not hybridized. In one example, the 5′-end of the aptamer is complementary and hybridizes to the second nucleic acid molecule, and first nucleic acid molecule is complementary and hybridizes to the 3′-end of the aptamer, and the middle of the aptamer nucleic acid molecule is not hybridized (FIG. 5C). Thus, all three nucleotides are attached directly or indirectly to the solid support. One skilled in the art will appreciate that the orientation of the nucleic acid molecules can be reversed (e.g., attach 5′-end of the first nucleic acid to the solid support).

To determine whether the nucleic acid-GOx conjugate strand should hybridize to the middle of the aptamer, or to the 3′-end of the aptamer, labeled DNA-GOx conjugates can be used to identify the optimal location, using the methods described in the Examples below. As shown in FIGS. 5A and 5B, for Cocaine and adenosine aptamer, the nucleic acid-GOx conjugate hybridizes to the middle of the aptamer, since the 3′-end can have structure switching after the target binding. But for IFN-γ, FIG. 5C, the nucleic acid-GOx conjugate hybridizes to the aptamer 5′-end. IFN-γ is a cytokine related to human immune system, and IFN-γ release assay is currently used for the diagnosis of tuberculosis. Thus, the disclosed methods and sensors can be used to diagnose tuberculosis.

Binding of the target to the aptamer nucleic acid molecule results in a conformational change in the aptamer nucleic acid molecule (such as the 3′-end). This conformational change results in displacement of the second nucleic acid molecule (nucleic acid-GOx conjugate) from the aptamer nucleic acid molecule, and from the solid support, thereby releasing the GOx from the solid support (or allowing it to travel to another region of the solid support) (right hand side of FIGS. 5A-5C). Known aptamers can be used, and modified for the methods and sensors provided herein.

For example, FIGS. 5A-5C show how a cocaine, adenosine, and IFN-γ aptamer can be modified and attached to a solid support. For example, the 5′-end of the aptamer can be extended to generate a sequence that is complementary to the anchor nucleic acid molecule (such as at least 90%, at least 91%, at least 92%, at least 93%, at least 94%, at least 95%, or 100% complementarity). For example, the 5′-end of the aptamer can be extended by at least 5 nt, at least 6 nt, at least 7 nt, at least 8 nt, at least 9 nt, at least 10 nt, at least 11 nt, at least 12 nt, at least 15 nt, at least 18 nt, at least 21 nt, or at least 22 nt, such as 6 nt-25 nt, 9 nt-22 nt, or 15 nt-22 nt, wherein this added sequence is designed to be complementary to the first nucleic acid strand (anchoring nucleic acid) attached to the solid support.

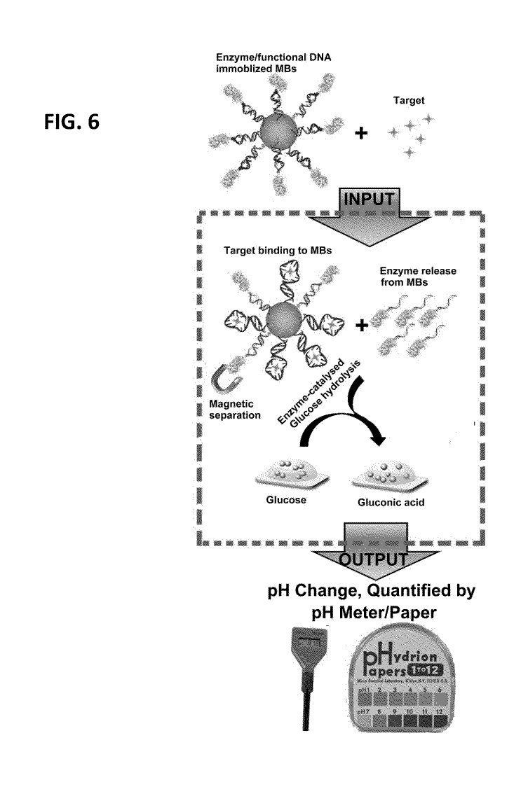

In some examples, the solid support is a bead. As shown in FIG. 6, after the GOx is released from the beads into a solution, the beads and the solution containing the GOx can be separated, for example by centrifugation, or by using a magnet if the beads are magnetic. The resulting solution can be contacted with glucose (e.g., glucose added) to allow the formation of gluconic acid, and the pH of the solution determined.

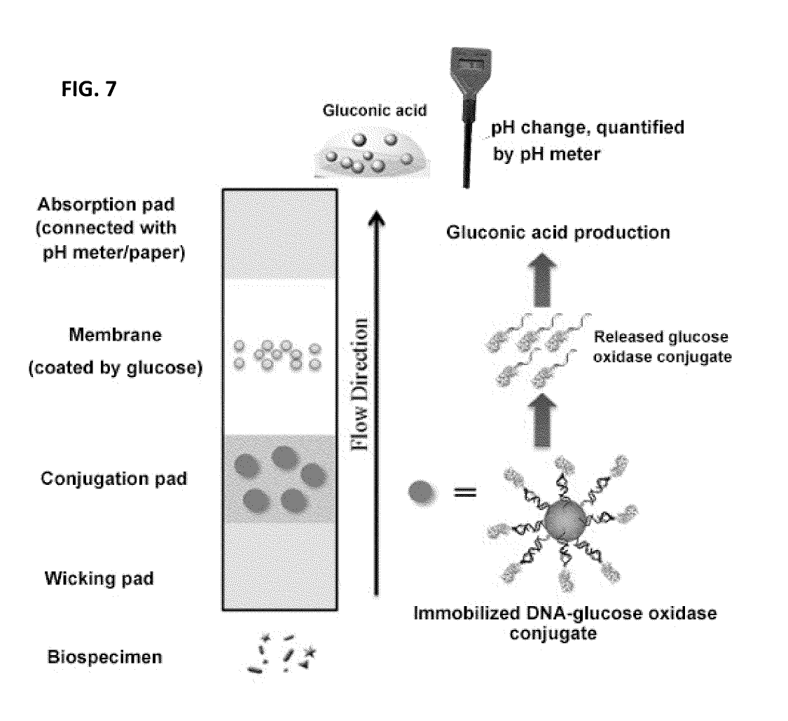

In some examples, the solid support is a lateral flow device (e.g., see FIG. 7). In such examples, the sample can be applied to the device, for example at an application pad, and the sample allowed to travel or flow through the device. One region of the lateral flow device includes immobilized anchoring nucleic acid molecule, nucleic acid-GOx conjugate (e.g., DNA-GOx), as well as the aptamer recognition molecule specific for the target, for example on a conjugation pad. These nucleic acid molecules can be attached to beads, and the bead immobilized onto the conjugation pad. The target in the sample (if present) flows through the lateral flow device and binds to the aptamer molecule on the lateral flow device, thereby forming a target-aptamer complex. After formation of the target-aptamer complex, this will result in a conformational change in the structure of the aptamer. As a result of this conformational change, the nucleic acid-GOx complex attached to the lateral flow device is released from the conjugation pad (or beads on the conjugation pad), and can to flow to another region of the lateral flow device, such as a membrane that includes glucose, under conditions that permit the formation of gluconic acid. Thus, in some examples, separating the solid support from the released GOx means that the released GOx is allowed to flow to a different part of the lateral flow device, such as a membrane that includes glucose. The resulting gluconic acid can change the pH of a solution, which can be detected with a pH meter or pH paper. In some examples, the lateral flow strip includes pH paper, which can be used as a read-out of pH. In some examples, the lateral flow strip includes pH paper (for example the absorption pad can be pH paper), which can be used as a read-out of pH. In some examples, the resulting droplet is read by a pH meter.

A specific exemplary lateral flow device is shown in FIG. 7. The lateral flow device includes a bibulous lateral flow strip, which can be present in housing material (such as plastic or other material). The lateral flow strip is divided into a proximal wicking pad, a conjugation pad (containing immobilized anchoring nucleic acid molecule, nucleic acid-GOx conjugate (e.g., DNA-GOx), and the aptamer recognition molecule specific for the target, which may be present on beads as shown), a membrane coated with glucose, and a distal absorption pad (which can be connected with pH paper or a pH meter). The flow path along strip passes from proximal wicking pad, through the conjugation pads, into the membrane coated with glucose, for eventual collection in absorption pad.

In operation of the particular embodiment of a lateral flow device illustrated in FIG. 7, a fluid sample containing a target of interest (or suspected of containing such), such as a metal target agent, is applied to the wicking pad, for example dropwise or by dipping the end of the device into the sample. If the sample is whole blood, an optional developer fluid can be added to the blood sample to cause hemolysis of the red blood cells and, in some cases, to make an appropriate dilution of the whole blood sample. From the wicking pad, the sample passes, for instance by capillary action, to the conjugation pad. In the conjugation pad, the target of interest binds the immobilized aptamer. For example, if the aptamer is specific for adenosine, adenosine in the sample will bind to the immobilized aptamer contained in the conjugation pad. After this binding, a target-aptamer complex is formed, resulting in a conformational change in the structure of the aptamer. This conformational change causes release of the nucleic acid-GOx conjugate (e.g., DNA-GOx) (shown as “release glucose oxidase conjugate”), which is allowed to flow to the membrane containing glucose, where the GOx can interact with glucose present on the membrane, thereby producing gluconic acid. The resulting gluconic acid can subsequently flow to the absorption pad, which can be read by a pH meter or contacted with pH paper, wherein detection of a decrease in the pH indicates the presence of target agent in the sample tested.

In some examples, the solid support is a microfluidic device (e.g., see FIG. 8). In examples where the recognition molecule is an aptamer, the test sample is introduced into the microfluidic device and mixed with droplets of buffer reagents (such as red blood cell lysis buffers and suitable buffers for the enzymatic reaction) and starting products. In an example the one or more starting products includes the aptamer, and nucleic acid-GOx conjugate. The mixture droplet moves into a first mixing chamber for sufficient time to ensure that the aptamer can bind the target, and that the aptamer conformation is changed, releasing the GOx from the beads or grapheme oxide. The GOx can be released from the first mixing chamber A, and if desired, can travel through a filter to remove undesired reagents. The released GOx is allowed to react with glucose, which can be in the second mixing chamber B or can be between the filter and the mixing chamber, to form gluconic acid, for example in a second mixing chamber, under conditions that allow for completion of the enzymatic reaction and the gluconic acid to be released from the second mixing chamber. Finally, the droplet containing gluconic acid is tested by a pH meter or pH paper after it is released from the microfluidic device. In some examples, the gluconic acid is mixed with a solution prior and the pH of the solution determined.

Another Example when Recognition Molecule is an Aptamer

In one example, the solid support used in the method includes an aptamer recognition molecule specific for the target. A thiolated aptamer is conjugated to GOx using heterobifunctional linker sulfo-SMCC, wherein the aptamer-GOx conjugate is attached to the solid support by non-covalent assembly of aptamers on a graphene oxide (GO) surface which is induced by π-π stacking of DNA bases on GO. Binding of the target to the aptamer results in a conformational change in the nucleic acid molecule and displaces the nucleic acid molecule having the GOx from the solid support, thereby releasing the GOx from the solid support. Known aptamers can be used, and modified for the methods and sensors provided herein. In one example, the aptamer includes a base spacer for conjugation, such as at least 6 nucleotides (nt), such as at least 7 nt, at least 8 nt, at least 9 nt, at least 10 nt, at least 11 nt, at least 12 nt, at least 13 nt, at least 14 nt, or at least 15 nt, such as 6 nt-20 nt, 6 nt-15 nt, 6 nt-12 nt, 9 nt-12 nt, for example 12 nt. In one example, the base spacer is a plurality of “A”s.

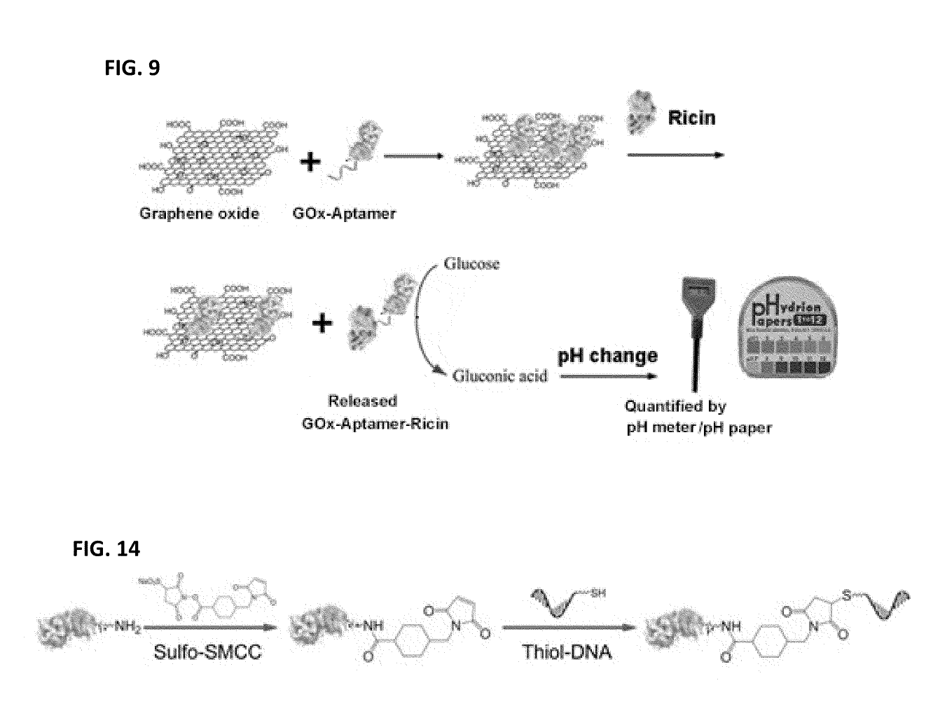

In some examples, the solid support is a graphene oxide. As shown in FIG. 9, after the GOx is released from the graphene oxide into a solution, the graphene oxide and the solution containing the GOx can be separated. The resulting solution can be contacted with glucose (e.g., glucose added) to allow the formation of gluconic acid, and the pH of the solution determined.

The graphene oxide molecules containing the aptamer-GOx conjugate can be used as part of a lateral flow device or a microfluidic device as describe above for other aptamers.

### Exemplary Competitive Assays

Turn on and turn off competitive assay methods can be used to detect a target, such as a target of interest having only one binding site (e.g., some small molecular targets). Thus, in some examples a competitive assay method is used to detect a mono-epitope target. In one example of a competitive assay, the target in a sample competes with its target-GOx conjugate analogue to bind to a recognition molecule, such as an antibody, nucleic acid, DNAzyme, or aptamer.

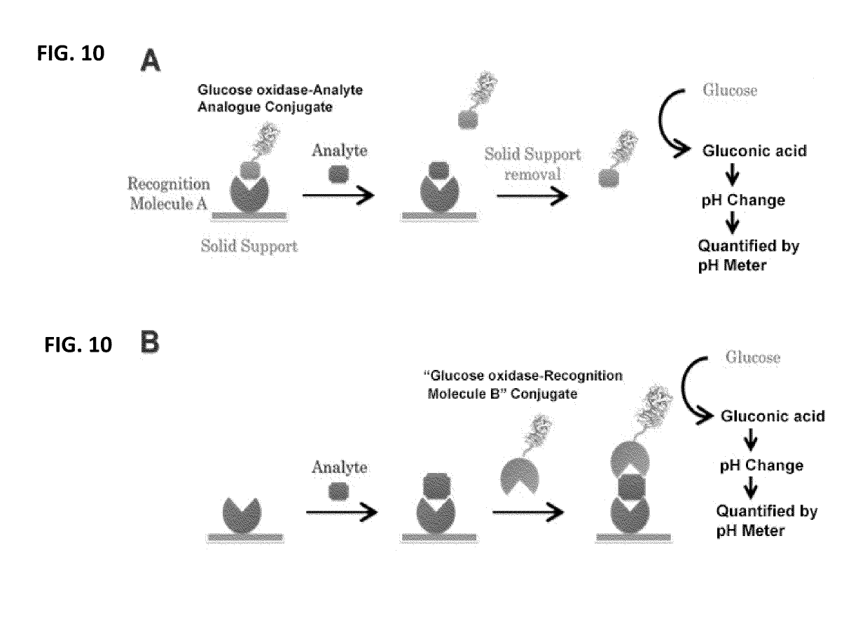

FIG. 10A provides an overview of such an example, also referred to as a release-based assay. In this method, the recognition molecule is bound or attached to both the solid support and to GOx-analyte (target) conjugate. The GOx that can catalyze the conversion of glucose into gluconic acid is conjugated with the target molecule or an analogue thereof using a conjugation method to form GOx-analyte conjugate (FIG. 10A). The target analogue can be any substance that can bind to the recognition molecule and complete with the binding between the target and the recognition molecule. Methods of attaching GOx to the recognition molecule (e.g., Ab or nucleic acid) are routine, and can include conjugating biotin with GOx (e.g., using biotin labeling kits), and the resulting biotin-GOx attached to a biotinylated antibody by adding streptavidin as a linker. Exemplary kits are commercially available, and include the FastLink Glucose Oxidase Labeling Kit (Abnova). Commercialized GOx labeled antibodies are also available. Thus, the GOx-analyte conjugate binds to the solid support through the interaction between GOx-analyte conjugate and the recognition molecule. When samples containing the target are applied to or contacted with the solid support, the GOx-analyte conjugate will be released as a result of the competition between GOx-analyte conjugate and target agent in the sample, for binding with recognition molecule. The more target present in the sample, the less GOx-analyte conjugates remain bound to the recognition molecule and yield corresponding changes in the signal readout. The concentration of GOx-analyte conjugate released can be proportional to the target concentration in the sample. After removal of the solid support or separation of the unbound target-glucose oxidase conjugate, GOx-analyte conjugate remaining in the solution can catalyze the conversion of glucose substrate into gluconic acid, which is detected by a pH meter or pH paper, and the readout can be proportional to the concentration or amount of target in the sample. This is a turn “on” version of the method because the more target in the sample, the more analyte-GOx conjugate present in the solution, and the greater the decrease in pH detected, and vice versa. However, one skilled in the art will appreciate that if instead the separated solid support is used for gluconic acid production in a glucose solution, the situation reverses, thus providing a turn “off” method that gives less pH change for the samples containing more targets (as there is less GOx-analyte conjugate remaining on the solid support) and vice versa.

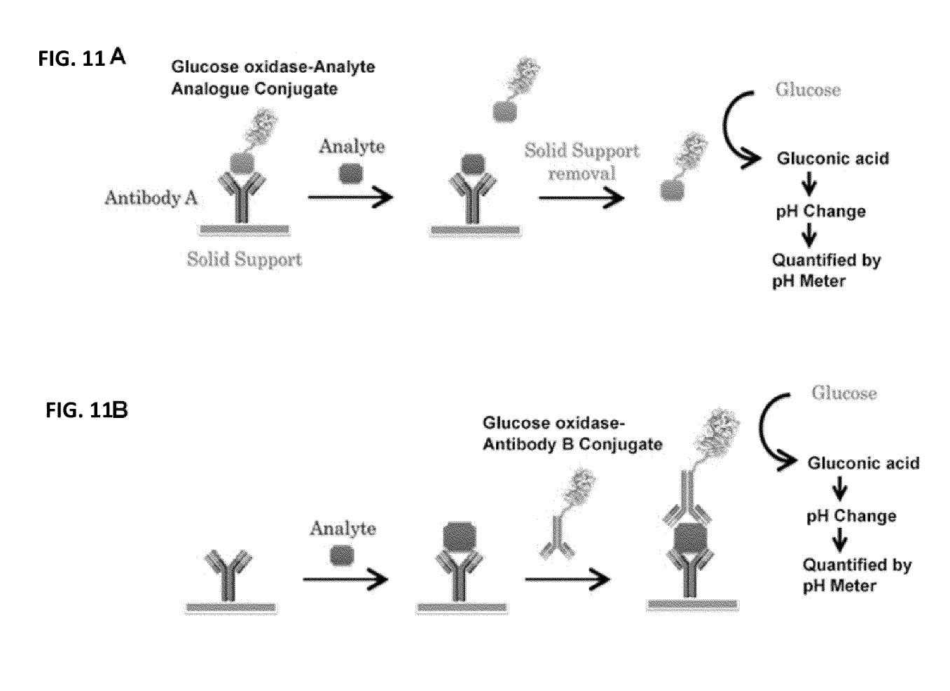

As shown in FIG. 11A, the recognition molecule in FIG. 10A can be an antibody. Thus, any target agent for which specific antibodies are available can be quantified using the methods provided herein, of example by using a pH meter or pH paper. As shown in FIG. 11A, the antibody is immobilized on the solid support using routine conjugation methods. The GOx-analyte conjugate is added and will bind to the antibody. The GOx-analyte conjugate can be prepared using routine methods. A sample containing analyte (e.g., suspected of containing the target agent) is contacted with the solid support under conditions that permit the target to specifically bind to the antibody, thereby displacing the GOx-analyte conjugate due to competition. The amount of GOx-analyte conjugate released can be proportional to the concentration of target in the sample. After removal of the solid support, the GOx-analyte conjugate can convert the glucose into gluconic acid, which can change the pH of the solution, and which can be detected by a pH meter or pH paper, and the readout can be proportional to the concentration or amount of target in the sample. This is a turn “on” version of the method because the more target in the sample, the more GOx-analyte conjugate present in the solution, and the greater the decrease in pH detected, and vice versa. However, one skilled in the art will appreciate that if instead the separated solid support is used for gluconic acid production in a glucose solution, the situation reverses, thus providing a turn “off” method that gives less pH change for the samples containing more targets (as there is less GOx-analyte conjugate remaining on the solid support) and vice versa.

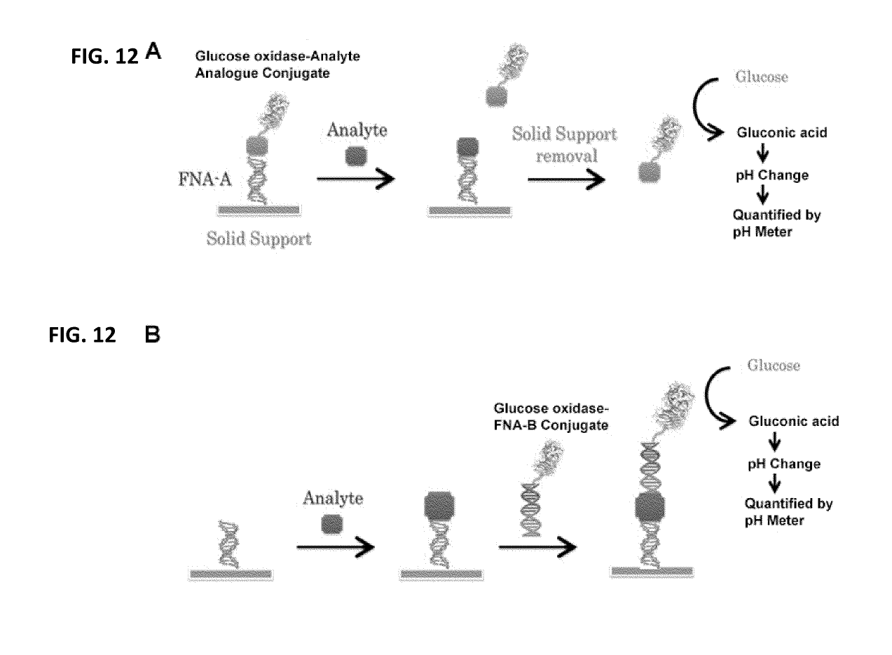

As shown in FIG. 12A, the recognition molecule in FIG. 10A can be a nucleic acid, such as a functional nucleic acid, FNA (e.g., aptamer, DNAzyme, or aptazyme). Thus, any target agent for which a specific FNA is available can be quantified using the methods provided herein, of example by using a pH meter or pH paper. As shown in FIG. 12A, the FNA is immobilized on the solid support using routine immobilization methods. The GOx-analyte analogue conjugate is added and will bind to the FNA. The GOx-analyte conjugate can be prepared using routine methods. A sample containing analyte (e.g., suspected of containing the target agent) is contacted with the solid support under conditions that permit the target agent to specifically bind to FNA, thereby displacing the GOx-analyte conjugate due to competition. The amount of GOx-analyte conjugate released can be proportional to the concentration of target agent in the sample. After removal of the solid support, the GOx-analyte conjugate can convert the glucose into gluconic acid, which is detected by a pH meter or pH paper, and the readout can be proportional to the concentration or amount of target in the sample. This is a turn “on” version of the method because the more target in the sample, the more GOx-analyte conjugate present in the solution, and the greater the decrease in pH detected, and vice versa. However, one skilled in the art will appreciate that if instead the separated solid support is used for gluconic acid production in a glucose solution, the situation reverses, thus providing a turn “off” method that gives less pH change for the samples containing more targets (as there is less GOx-analyte conjugate remaining on the solid support) and vice versa.

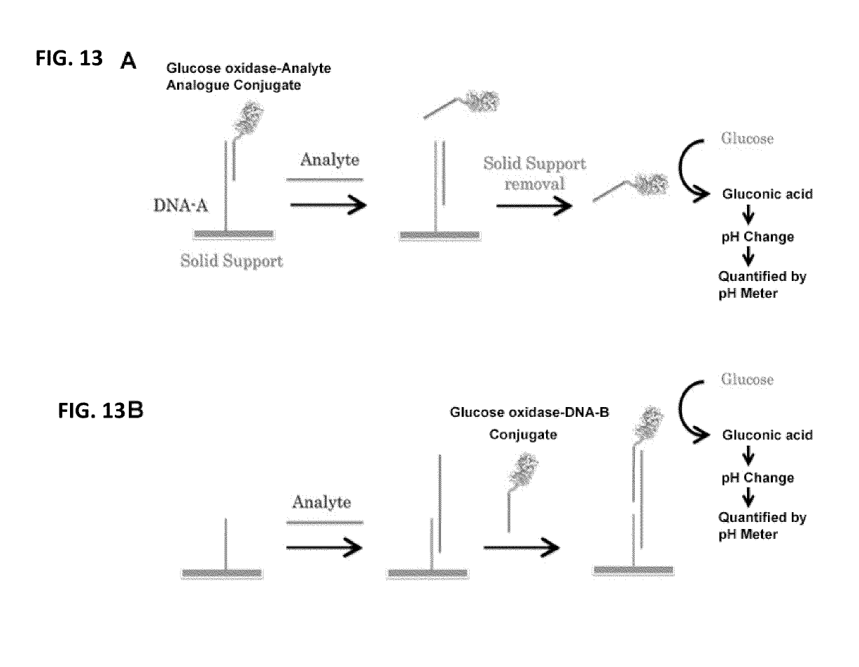

Because the target can be any species that can be recognized by the recognition molecules shown in FIG. 10A, the disclosure is not limited to the use of a particular recognition component. For example, in addition to antibodies (FIG. 11A), and functional nucleic acids (FIG. 12A), they may include peptides, proteins, polymers and even small molecules that recognize targets analytes. For example, as shown in FIG. 13A, nucleic acids can be detected by hybridization between nucleic acids. In this example, the target agent is a nucleic acid, and the recognition molecule of FIG. 10A and is a nucleic acid molecule (e.g., DNA) that can hybridize with the analyte. As shown in FIG. 13A, DNA (or RNA) is immobilized on the solid support using routine immobilization methods. The GOx-analyte conjugate is added and will bind to the immobilized DNA. The GOx-analyte conjugate can be prepared using routine methods. A sample containing analyte (e.g., suspected of containing the target agent) is contacted with the solid support under conditions that permit the target nucleic acid to specifically bind to the immobilized DNA, thereby displacing the GOx-analyte conjugate due to competition. The amount of GOx-analyte conjugate released can be proportional to the concentration of target agent in the sample. After removal of the solid support, the GOx-analyte conjugate can convert the glucose into gluconic acid, which is detected by a pH meter or pH paper, and the readout can be proportional to the concentration or amount of target in the sample. This is a turn “on” version of the method because the more target in the sample, the more GOx-analyte conjugate present in the solution, and the greater the decrease in pH detected, and vice versa. However, one skilled in the art will appreciate that if instead the separated solid support is used for gluconic acid production in a glucose solution, the situation reverses, thus providing a turn “off” method that gives less pH change for the samples containing more targets (as there is less GOx-analyte conjugate remaining on the solid support) and vice versa.

The competitive assays can be performed with the sensors provided herein, such as a lateral flow strip or a microfluidic device, essentially as described herein. For example, the recognition molecule-target analogue-GOx complex can be present on a solid substrate, such as magnetic beads (MBs), such as amine-modified magnetic beads, as well as array plates (such as an ELISA plate), lateral flow devices (e.g., on a conjugation pad, which may include beads), and microfluidic devices. The test sample is incubated with the recognition molecule-target analogue-GOx complex, under conditions that allow the target (if present) to bind to the complex and release the GOx, (which in some examples can travel to another part of the device, such as to a membrane that includes glucose, or can be removed for example by removing solution from a well of a multi-well plate, or using a magnet to remove magnetic beads from solution). The solid substrate or the solution containing the released GOx can contacted with glucose, and pH detected, and a determination made as to whether the target is present or absent in the sample by correlating the pH detected. For example, if the target is present, there will be a significant decrease in the pH of the solution that contained the GOx, but not if the target-recognition molecule-solid substrate complex is used to detect glucose. In contrast, if the target is absent, there will be low or no significant decrease in the pH of the solution that contained the GOx but will be significant if the target-recognition molecule-solid substrate complex is used to detect pH.

### Exemplary Sandwich Assays

In some examples, sandwich assay methods can be used to detect a target, such as a target of interest having numerous binding sites (e.g., some large molecular targets). Thus, in some examples a sandwich assay method is used to detect a multi-epitope target. In one example of a sandwich assay, a first recognition molecule attached to a solid substrate binds to the target in a sample, resulting in a first-recognition molecule-target complex, and a second recognition molecule attached to GOx binds to the first-recognition molecule-target complex, forming a sandwich complex of first-recognition molecule-target-second recognition molecule-GOx. Examples of recognition molecules include but are not limited to an antibody, nucleic acid, DNAzyme, or aptamer.

FIG. 10B provides an overview of such an example. In this method, a first recognition molecule (blue) and a second recognition molecule (green) (referred to herein as the recognition molecule that can bind to the target agent with high specificity) can be the same or different molecules, wherein both can bind to the analyte (target). In one example the first recognition molecule and the second recognition molecule are different molecules (such as one is an antibody and the other is a FNA), but both specifically bind to the target. The GOx that can catalyze the conversion of a glucose into gluconic acid is attached to the second recognition molecule (FIG. 10B) using a conjugation method to form the GOx-second recognition molecule conjugate. Initially, the first recognition molecule is immobilized to the solid support. When a sample containing or suspected of containing the target (analyte) is applied to solid support, the analyte binds to the first recognition molecule, forming first recognition molecule-target complex. Subsequently, the GOx-second recognition molecule conjugate is added and will bind to the target on the first recognition molecule, forming a sandwich structure (first-recognition molecule-target-second recognition molecule-GOx). The amount of GOx-second recognition molecule conjugate bound to the target (and thus the solid support) can be proportional to the concentration of target in the sample. After applying glucose to the solid support, the bound GOx can convert glucose into gluconic acid, and the gluconic acid produced can change the pH of a solution, which can be detected by a pH meter or pH paper (and in some examples quantified). The readout is proportional to the amount of target in the sample. The target can be any substance that can be recognized by the first and second recognition molecules.

As shown in FIG. 11B, the recognition molecules in FIG. 10B can be antibodies. Thus, any target agent for which specific antibodies are available can be quantified using the methods provided herein, of example by using a pH meter or pH paper. As shown in FIG. 11B, the antibodies can both bind the analyte (target); they can be the same antibody or different antibodies that are specific for the same analyte. As shown in FIG. 11B, the first antibody is immobilized on the solid support using routine conjugation methods. A sample containing analyte (e.g., suspected of containing the target) is contacted with the solid support under conditions that permit the target agent to specifically bind to the first antibody. GOx-second-antibody conjugate is added and will bind to the analyte (target) bound to the first antibody, forming a sandwich structure. The GOx-second-antibody conjugate can be prepared using routine methods. The amount of GOx-second-antibody conjugate bound can be proportional to the concentration or amount of target in the sample. After applying a glucose solution to the solid support, the bound GOx-second-antibody conjugate can convert the glucose into gluconic acid, which can change the pH of the solution, and can be detected by a pH meter or pH paper. The readout can be proportional to the concentration or amount target in the sample tested.

As shown in FIG. 12B, the recognition molecules in FIG. 10B can be functional nucleic acids, FNA (e.g., aptamer, DNAzyme, or aptazyme). Thus, any target agent for which a specific FNA is available can be quantified using the methods provided herein, of example by using a pH meter or pH paper. As shown in FIGS. 3A and 3B, first and second FNAs both can bind the analyte (target); they can be the same FNA or different FNAs that are specific for the same analyte. As shown in FIG. 12B, the first FNA is immobilized on the solid support using routine methods. A sample containing analyte (e.g., suspected of containing the target agent) is contacted with the solid support under conditions that permit the target to specifically bind to the first FNA, thereby forming a first FNA-target complex. Subsequently, the GOx-second FNA conjugate is added and will bind to the target on the first recognition molecule, forming a sandwich structure (first FNA-target-second FNA-GOx). The amount of GOx-second FNA conjugate bound to the target (and thus the solid support) can be proportional to the concentration of target in the sample. After applying glucose to the solid support, the bound GOx can convert glucose into gluconic acid, and the gluconic acid produced can change the pH of a solution, which can be detected by a pH meter or pH paper (and in some examples quantified). The readout is proportional to the amount of target in the sample.

Because the target can be any species that can be recognized by the recognition molecules shown in FIG. 10B, the disclosure is not limited to the use of a particular recognition component. For example, in addition to antibodies (FIG. 11B), and functional nucleic acids (FIG. 12B), they may include peptides, proteins, polymers and even small molecules that recognize targets analytes. For example, as shown in FIG. 13B, nucleic acids can be detected by hybridization between nucleic acids. In this example, the target agent is a nucleic acid, and the recognition molecule of FIG. 10B and is a nucleic acid molecule (e.g., DNA) that can hybridize with the analyte. As shown in FIG. 13B, first DNA (or RNA) is immobilized on the solid support using routine immobilization methods. A sample containing analyte (e.g., suspected of containing the target) is contacted with the solid support under conditions that permit the target agent to specifically bind to the first DNA or RNA, thereby forming a target-first DNA (or RNA) complex. The GOx-second DNA (or RNA) conjugate is added and will bind to the immobilized DNA. Subsequently, the GOx-second DNA (or RNA) conjugate is added and will bind to the target on the first DNA (or RNA), forming a sandwich structure (first DNA (or RNA)-target-second DNA (or RNA)-GOx). The amount of GOx-second DNA (or RNA) conjugate bound to the target (and thus the solid support) can be proportional to the concentration of target in the sample. After applying glucose to the solid support, the bound GOx can convert glucose into gluconic acid, and the gluconic acid produced can change the pH of a solution, which can be detected by a pH meter or pH paper (and in some examples quantified). The readout is proportional to the amount of target in the sample.

The sandwich assays can be performed with the sensors provided herein, such as a lateral flow strip or a microfluidic device, essentially as described herein. For example, the first recognition molecule can be present on a solid substrate, such as magnetic beads (MBs), such as amine-modified magnetic beads, as well as array plates (such as an ELISA plate), lateral flow devices (e.g., on a conjugation pad, which may include beads), and microfluidic devices. The test sample is incubated with the first recognition molecule, under conditions that allow the target (if present) to bind to the first recognition molecule, thereby generating a target-first recognition molecule complex. This complex is contacted with a second recognition molecule-GOx complex, which may be added to the well of a plate, or may be present on a conjugation pad, may be added to a lateral flow strip, or may be part of a microfluidic device. If the target is present, the second recognition molecule-GOx complex will bind to the target-first recognition molecule complex. Unbound materials can be separated or removed. The resulting complex can be contacted with glucose, which may be added to the well of a plate, may be present on a membrane, may be added to a lateral flow strip, or may be part of a microfluidic device. The pH is detected, and a determination made as to whether the target is present or absent in the sample by correlating the pH detected.

### Samples

Any biological or environmental specimen that may contain (or is known to contain or is suspected of containing) a target agent can be used in the methods herein.

Biological samples are usually obtained from a subject and can include genomic DNA, RNA (including mRNA), protein, or combinations thereof. Examples include a tissue or tumor biopsy, fine needle aspirate, bronchoalveolar lavage, pleural fluid, spinal fluid, saliva, sputum, surgical specimen, lymph node fluid, ascites fluid, peripheral blood (such as serum or plasma), urine, saliva, buccal swab, and autopsy material. Techniques for acquisition of such samples are well known in the art (for example see Schluger et al. J. Exp. Med. 176:1327-33, 1992, for the collection of serum samples). Serum or other blood fractions can be prepared in the conventional manner. Samples can also include fermentation fluid and tissue culture fluid.

Environmental samples include those obtained from an environmental media, such as water, air, soil, dust, wood, plants, or food (such as a swab of such a sample). In one example, the sample is a swab obtained from a surface, such as a surface found in a building or home. In one example the sample is a food sample, such as a meat, dairy, fruit, or vegetable sample. For example, using the methods provided herein, adulterants in food products can be detected, such as a pathogen or toxin or other harmful product.

In other examples, a sample includes a control sample, such as a sample known to contain or not contain a particular amount of the target.

Once a sample has been obtained, the sample can be used directly, concentrated (for example by centrifugation or filtration), purified, liquefied, diluted in a fluid, or combinations thereof. In some examples, proteins or nucleic acids or pathogens are extracted from the sample, and the resulting preparation (such as one that includes isolated DNA, RNA, and/or proteins) analyzed using the methods provided herein.

### Sensors for Detecting Target Agents

Provided herein are sensors that can be used to detect an analyte of interest (referred to herein as a target). Such sensors can be engineered using the methods provided herein to detect a broad range of targets, significantly facilitating rational design and increasing the efficiency of sensor development. By combining molecules that can specifically bind to a target agent (referred to herein as recognition molecules), GOx that can convert a glucose into gluconic acid, and commercially available pH meters and pH paper, a general platform for the design of portable, low-cost and quantitative sensors specific to a broad range of analytes is provided. In one example, the approach is based on the target agent-induced release of the GOx from a solid support, or the use of an GOx-recognition molecule complex that can also bind to the target agent, wherein the GOx can convert a glucose into gluconic acid, which can decrease the pH of a solution, which can be detected.

Disclosed herein are sensors that permit detection of a target agent. In one example, such sensors include a solid support to which is attached a recognition molecule that permits detection of a target agent. For example, the recognition molecule can bind to the target agent with high specificity in the presence of the target agent but not significantly to other agents. The sensors in some examples also include GOx, such as a nucleic acid-GOx conjugate, that can catalyze the conversion of a glucose into gluconic acid In one example, the GOx is attached to the recognition molecule that permits detection of a target agent, such that in the presence of the target agent, GOx is released from the solid support and can convert the glucose into gluconic acid, which can be detected by a change in pH. In one example, the GOx is not attached to the recognition molecule, but in the presence of the target agent the recognition molecule is cleaved, resulting in GOx release from the solid support, converting glucose into gluconic acid, which can be detected by a change in pH. In another example, the GOx is not initially part of the sensor, but instead after the target agent binds to the recognition molecule, a second recognition molecule (which may be the same or a different recognition molecule attached to the solid support) which has conjugated thereto the GOx, binds to the target agent bound to the first recognition molecule bound to the solid support, thus creating a type of “sandwich.” The bound GOx can then convert glucose into gluconic acid.

One skilled in the art will recognize that any approach using other techniques to transform one target agent's concentration information into another's, which is subsequently detected using the methods in this application, can be used. For example, if target agent A can quantitatively produce substance B by a certain technique, one can simply use the methods in this application to detect substance B, and then convert the concentration of substance B into that of target agent A in the sample.

In one example, the sensor includes a solid support. The solid support can include a first nucleic acid molecule having a 5′-end and a 3′-end, wherein the first nucleic acid is attached to the solid support by one end (such as the 5′-end), and wherein the first nucleic acid is complementary to a 5′-end of a substrate strand of a DNAzyme specific for a target that can be detected by the sensor. The solid support also includes a second nucleic acid molecule, referred to herein as the nucleic acid-GOx conjugate, or the DNA-GOx conjugate, which has a 5′-end and a 3′-end, wherein the 5′-end of the second nucleic acid molecule is hybridized to the 3′-end of the first nucleic acid molecule and wherein the 3′-end of the second nucleic acid molecule has GOx attached.

In one example, the solid support can include a first nucleic acid molecule having a 5′-end and a 3′-end, wherein the first nucleic acid is attached to the solid support by one end (such as the 3′-end); a second nucleic acid molecule (referred to herein as the nucleic acid-GOx conjugate, or the DNA-GOx conjugate,) having a 5′-end and a 3′-end, wherein the 3′-end of the second nucleic acid molecule is proximal (e.g., hybridized or attached) to the 5′-end of the first nucleic acid molecule and wherein the 5′-end of the second nucleic acid molecule has GOx attached (or vice versa); and an aptamer specific for a target that can be detected by the sensor, wherein the aptamer comprises a nucleic acid molecule having a 5′-end and a 3′-end, wherein the aptamer nucleic acid molecule is complementary and hybridizes to the first nucleic acid molecule and to the second nucleic acid molecule, wherein the 3′-end of the aptamer nucleic acid molecule is in some examples not hybridized.

In one example, the solid support can include an aptamer nucleic acid molecule having a first end and a second end, wherein the nucleic acid molecule is attached to the solid support by the first end and comprises glucose oxidase on the second end, and wherein the solid support comprises graphene oxide.

In one example, the solid support can include a recognition molecule bound to a target-glucose oxidase complex, wherein in the presence of the target in a sample the amount of target-glucose oxidase complex bound to the solid support decreases, and wherein the amount of target in the sample is proportional to the amount of unbound target-GOx complexes.

### Solid Supports

The solid support which forms the foundation of the sensor can be formed from known materials, such as any water immiscible material. In some examples, suitable characteristics of the material that can be used to form the solid support surface include: being amenable to surface activation such that upon activation, the surface of the support is capable of covalently attaching a recognition molecule that can bind to the target agent with high specificity, such as an oligonucleotide or a protein; being chemically inert such that at the areas on the support not occupied by the molecule can bind to the target agent with high specificity are not amenable to non-specific binding, or when non-specific binding occurs, such materials can be readily removed from the surface without removing the molecule can bind to the target agent with high specificity.

A solid phase can be chosen for its intrinsic ability to attract and immobilize an agent, such as recognition molecule that can bind to the target agent with high specificity. Alternatively, the solid phase can possess a factor that has the ability to attract and immobilize an agent, such as a recognition molecule. The factor can include a charged substance that is oppositely charged with respect to, for example, the recognition molecule itself or to a charged substance conjugated to the recognition molecule. In another embodiment, a specific binding member may be immobilized upon the solid phase to immobilize its binding partner (e.g., a recognition molecule). In this example, therefore, the specific binding member enables the indirect binding of the recognition molecule to a solid phase material.

The surface of a solid support may be activated by chemical processes that cause covalent linkage of an agent (e.g., a recognition molecule specific for the target agent) to the support. However, any other suitable method may be used for immobilizing an agent (e.g., a recognition molecule) to a solid support including, without limitation, ionic interactions, hydrophobic interactions, covalent interactions and the like. The particular forces that result in immobilization of a recognition molecule on a solid phase are not important for the methods and devices described herein.

In one example the solid support is a particle, such as a bead. Such particles can be composed of metal (e.g., gold, silver, platinum), metal compound particles (e.g., zinc oxide, zinc sulfide, copper sulfide, cadmium sulfide), non-metal compound (e.g., silica or a polymer), as well as magnetic particles (e.g., iron oxide, manganese oxide). In some examples the bead is a latex or glass bead. The size of the bead is not critical; exemplary sizes include 5 nm to 5000 nm in diameter. In one example such particles are about 1 μm in diameter.

In another example, the solid support is a bulk material, such as a paper, membrane, porous material, water immiscible gel, water immiscible ionic liquid, water immiscible polymer (such as an organic polymer), and the like. For example, the solid support can comprises a membrane, such as a semi-porous membrane that allows some materials to pass while others are trapped. In one example the membrane comprises nitrocellulose. In a specific example the solid support is part of a lateral flow device that includes a region containing the sensors disclosed herein.

In some embodiments, porous solid supports, such as nitrocellulose, are in the form of sheets or strips, such as those found in a lateral flow device. The thickness of such sheets or strips may vary within wide limits, for example, at least 0.01 mm, at least 0.1 mm, or at least 1 mm, for example from about 0.01 to 5 mm, about 0.01 to 2 mm, about 0.01 to 1 mm, about 0.01 to 0.5 mm, about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof).

In yet other examples, the solid support is a material containing, such as a coating containing, any one or more of or a mixture of the ingredients provided herein.

A wide variety of solid supports can be employed in accordance with the present disclosure. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

The solid support can be any format to which the molecule specific for the test agent can be affixed, such as microtiter plates, multiwell plates, ELISA plates, test tubes, inorganic sheets, dipsticks, lateral flow devices, microfluidic devices, and the like. One example includes a linear array of molecules specific for the target agent, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range.

In one example the format is a bead, such as a silica bead or magnetic bead. In another example the format is a nitrocellulose membrane. In another example the format is filter paper. In yet another example the format is a glass slide. In one example, the solid support includes graphene oxide. In one example, the solid support is a polypropylene thread. One or more polypropylene threads can be affixed to a plastic dipstick-type device; polypropylene membranes can be affixed to glass slides.

In one example the solid support is a microtiter plate. For example sensors can be affixed to the wells of a microtiter plate (for example wherein some wells can contain a sensor to detect target X, while other wells can contain a sensor to detect target Y; or several wells might include the same sensor, wherein multiple samples can be analyzed simultaneously). The test sample potentially containing an analyte of interest can be placed in the wells of a microtiter plate containing a sensor disclosed herein, and the presence of the target detected using the methods provided herein in. One advantage of the microtiter plate format is that multiple samples can be tested simultaneously (together with controls) each in one or more different wells of the same plate; thus, permitting high-throughput analysis of numerous samples.

In some examples, the disclosed sensor is attached to more than one solid support. For example, a sensor containing a recognition molecule and/or a nucleic acid GOx-complex can be attached to a bead or to graphene oxide, which can then be attached to a conjugation pad of a lateral flow device or can be part of a microfluidic device.

Each of the supports and devices discussed herein (e.g., ELISA, lateral flow device, microfluidic device) can be, in some embodiments, formatted to detect multiple analytes by the addition of recognition molecules specific for the other analytes of interest. For example, certain wells of a microtiter plate can include recognition molecules specific for the other analytes of interest. Some lateral flow and microfluidic device embodiments can include secondary, tertiary or more capture areas containing recognition molecules specific for the other analytes of interest.

### Lateral Flow Devices

In one example, the solid support is a lateral flow device, which can be used to determine the presence and/or amount of one or more target agents in a fluid sample (or a sample suspended in a liquid, for example to transfer a target agent on a solid surface to the liquid). A lateral flow device is an analytical device having a test strip, through which flows a test sample fluid that is suspected of (or known to) containing a target. Based on the principles of a pregnancy strip lateral flow device, lateral flow devices that incorporate the disclosed sensors can be developed. In some examples, by using such as lateral flow devices, samples can be directly contacted with or applied to the lateral flow device, and no further liquid transfer or mixing is required. Such devices can be used to detect target agents, for example qualitatively or quantitatively.

Lateral flow devices are commonly known in the art, and have a wide variety of physical formats. Any physical format that supports and/or houses the basic components of a lateral flow device in the proper function relationship is contemplated by this disclosure. In one example, the lateral flow devices disclosed in U.S. Pat. No. 7,799,554, Liu et al. (Angew. Chem. Int. Ed. 45:7955-59, 2006), Apilux et al. (Anal. Chem. 82:1727-32, 2010), Dungchai et al. (Anal. Chem. 81:5821-6, 2009), or Dungchai et al. (Analytica Chemica Acta 674:227-33, 2010) (all herein incorporated by reference) are used, such as one made using the Millipore Hi-Flow Plus Assembly Kit. There are a number of commercially available lateral flow type tests and patents disclosing methods for the detection of large analytes (MW greater than 1,000 Daltons) (see for example U.S. Pat. Nos. 5,229,073; 5,591,645; 4,168,146; 4,366,241; 4,855,240; 4,861,711; and 5,120,643; European Patent No. 0296724; WO 97/06439; and WO 98/36278). There are also lateral flow type tests for the detection of small-analytes (MW 100-1,000 Daltons) (see for example U.S. Pat. Nos. 4,703,017; 5,451,504; 5,451,507; 5,798,273; and 6,001,658).

The construction and design of lateral flow devices is very well known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Devices described herein generally include a strip of absorbent material (such as a microporous membrane), which can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific recognition molecule(s) and/or other reagents (such as a nucleic acid-GOx that can convert glucose into gluconic acid) required for the detection and/or quantification of the particular analyte being tested for. Thus these zones can be viewed as functional sectors or functional regions within the test device.

These devices typically include a sample application area and one or more separate target agent capture areas (conjugation pad) in which an immobilized sensor disclosed herein is provided which sensor includes a recognition molecule having a specific binding affinity for a target agent. For example, a lateral flow device containing at least two separate target agent capture areas (such as 2, 3, 4, 5, 6, 7, 8, 9, 10, 11, 12, 13, 14, 15 or more) can be used to detect a plurality of different target agents in a single sample. Any liquid (such as a fluid biological sample) applied in the sample application area flows along a path of flow from the sample application area to the capture area. Upon binding of the target agent to the recognition molecule, the released GOx can catalyze the conversion of glucose into gluconic acid. The GOx flows to a downstream membrane containing glucose. The glucose is converted to gluconic acid, which flows to a downstream absorbent pad, which can act as a liquid reservoir. The resulting gluconic acid on the lateral flow strip can decrease the pH of the solution, and the pH of the solution detected with a pH meter or pH paper, for example by insertion of the device into a pH meter or applying it to pH paper (or pH paper can be a part of the device.

In one example where a lateral flow device can detect multiple targets, the device includes a single wicking pad or sample application area, and multiple conjugation pads, membranes and absorption pads (such that each conjugation pad is associated with a particular membrane and absorption pad). For example, each conjugation pad can include a different recognition molecule specific for a particular target agent and/or a nucleic acid-GOx conjugate. Thus, the gluconic acid produced as a result of the target agent and present on each absorption pad can be used to determine the presence of a particular target agent.

In one example, the recognition molecule is a nucleic acid aptamer (such as a DNA aptamer) with high specificity for the target. In one example, the recognition molecule is a DNAzyme or RNAzyme with high specificity for the target. In another example, the recognition molecule is an antibody that is specific for the target. In another example, the recognition molecule is a nucleic acid molecule that is specific for the target. Ideally, recognition molecules are able to recognize targets with high sensitivity and selectivity. Such molecules are known, and can also be readily obtained using known methods.

The lateral flow device can include a wicking pad, conjugation pad (the region of a lateral flow device where the recognition molecule and/or the nucleic acid-GOx conjugate is immobilized), membrane, absorption pad, and combinations thereof. Such pads can abut one another or overlap, and can be attached to a backing. Thus, a lateral flow device can include a sample application area or wicking pad, which is where the fluid or liquid sample is introduced or applied. In one example, the sample may be introduced to the sample application area by external application, as with a dropper or other applicator. In another example, the sample application area may be directly immersed in the sample, such as when a test strip is dipped into a container holding a sample. In yet another example, the sample may be poured or expressed onto the sample application area. In some examples, multiple discrete binding partners can be placed on the strip (for example in parallel lines or as other separate portions of the device) to detect multiple target agents in the liquid. The test strips can also incorporate control indicators, which provide a signal that the test has adequately been performed, even if a positive signal indicating the presence (or absence) of an analyte is not achieved.

A lateral flow device may have more than one conjugation area, for example, a “primary conjugation area,” a “secondary conjugation area,” and so on. For example, a different capture reagent can be immobilized in the primary, secondary, or other conjugation areas. Multiple conjugation areas may have any orientation with respect to each other on the lateral flow substrate; for example, a primary conjugation area may be distal or proximal to a secondary (or other) conjugation area and vice versa. Alternatively, a primary conjugation area and a conjugation (or other) capture area may be oriented perpendicularly to each other such that the two (or more) conjugation areas form a cross or a plus sign or other symbol. For example, Apilux et al. (Anal. Chem. 82:1727-32, 2010), Dungchai et al. (Anal. Chem. 81:5821-6, 2009), and Dungchai et al. (Analytica Chemica Acta 674:227-33, 2010), provide exemplary lateral flow devices with a central sample area and one or more conjugation areas distal to the sample area, which provide independent test zones where independent reactions can occur (e.g., each test zone has a different recognition molecule, and can further include as a membrane that includes glucose that can be converted into gluconic acid and an absorption pad that receives the generated gluconic acid, wherein each absorption pad can be independently read by a pH meter or pH paper), for example that form a “Y”, cloverleaf, or spoke-wheel pattern.

A lateral flow device can include a membrane, such as one that includes the glucose, and an absorption pad that draws the sample across the conjugation pad(s) and membrane(s) by capillary action and collects it.

Exemplary materials that can be used for the components of a lateral flow device are shown in Table 1. However, one of skill in the art will recognize that the particular materials used in a particular lateral flow device will depend on a number of variables, including, for example, the analyte to be detected, the sample volume, the desired flow rate and others, and can routinely select the useful materials accordingly.

TABLE 1
Exemplary materials for a lateral flow device

| Component | Exemplary Material |
|---|---|
| Wicking Pad | Glass fiber |
| | Woven fibers |
| | Screen |
| | Non-woven fibers |
| | Cellulosic filters |
| | Paper |
| Conjugation Pad | Glass fiber |
| | Polyester |
| | Paper |
| | Surface modified polypropylene |
| Membrane | Nitrocellulose (including pure nitrocellulose and |
| | modified nitrocellulose) |
| | Nitrocellulose direct cast on polyester support |
| | Polyvinylidene fluoride |
| | Nylon |
| Absorption Pad | Cellulosic filters |
| | Paper |

Lateral flow devices can in one example be a one-step lateral flow assay in which a sample fluid is placed in a sample or wicking area on a bibulous strip (though, non bibulous materials can be used, and rendered bibulous by applying a surfactant to the material), and allowed to migrate along the strip until the sample comes into contact with a recognition molecule that interacts with a target agent in the liquid. After the target agent binds to the recognition molecule, the GOx is released (for example from a solid support), and allowed to interact with glucose, thereby generating gluconic acid indicating that the interaction has occurred, and that the target agent is present in the sample. The resulting pH decrease due to the presence of gluconic acid can be detected with a pH meter or pH paper.

The sample known or suspected of containing one or more target agents is applied to or contacted with the wicking pad (which is usually at the proximal end of the device, but can for example be at the center of the device for example when multiple conjugation pads are included to detect multiple targets), for instance by dipping or spotting. A sample is collected or obtained using methods well known to those skilled in the art. The sample containing the test agent to be detected may be obtained from any source. The sample may be diluted, purified, concentrated, filtered, dissolved, suspended or otherwise manipulated prior to assay to optimize the results. The fluid sample migrates distally through all the functional regions of the strip. The final distribution of the fluid in the individual functional regions depends on the adsorptive capacity and the dimensions of the materials used.

The wicking pad ensures that the sample moves through the device in a controllable manner, such that it flows in a unilateral direction. The wicking pad initially receives the sample, and can serve to remove particulates from the sample. Among the various materials that can be used to construct a sample pad (see Table 1), a cellulose sample pad may be beneficial if a large bed volume (e.g., 250 μl/cm²) is a factor in a particular application. In one example, the wicking pad is made of Millipore cellulose fiber sample pads (such as a 10 to 25 mm pad, such as a 15 mm pad). Wicking pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

After contacting the sample to the wicking pad, the sample liquid migrates from bottom to the top because of capillary force (or from the center outwards). The sample then flows to one or more conjugation pads, which serves to, among other things, hold the recognition molecule and the nucleic acid-GOx conjugate. The recognition molecule and the nucleic acid-GOx conjugate can be immobilized to conjugation pads by spotting (for example the recognition molecule and/or the nucleic acid-GOx conjugate can be suspended in water or other suitable buffer and spotted onto the conjugation pad and allowed to dry). In some examples, the recognition molecule and/or the nucleic acid-GOx conjugate are attached to beads or graphene oxide, which is adhered to one or more conjugation pads. The conjugation pad can be made of known materials (see Table 1), such as glass fiber, such as one that is 10 to 25 mm, for example 13 mm. When the sample reaches the conjugation pad, target agent present in the sample can bind to the recognition molecule, resulting in the release of the GOx from the conjugation pad (or a different conjugation pad). In a particular embodiment, the recognition molecule and/or the nucleic acid-GOx conjugate associated with the conjugation pad(s) is immobilized to a bead or graphene oxide.

The released GOx then flows to the membrane coated by glucose. Then, the released GOx catalyzes the production of gluconic acid from glucose in the membrane coated by glucose. The membrane portion can be made of known materials (see Table 1), such as a HiFlow Plus Cellulose Ester Membrane, such as one that is 10 to 40 mm, for example 25 mm. Methods that can be used to attach the glucose to the membrane include spotting (for example the one or other substance can be suspended in water or other suitable buffer and spotted onto the membrane and allowed to dry).

Finally, the gluconic acid produced in the membrane moves with the flow and reaches the absorption pad, where it changes the pH of the solution, whose pH is then detected. The absorbent pad acts to draw the sample across the conjugation pad and membrane by capillary action and collect it. This action is useful to insure the sample solution will flow from the sample or wicking pad unidirectionally through conjugation pad and the membrane to the absorption pad. Any of a variety of materials is useful to prepare an absorbent pad, see, for example, Table 1. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad. In one example the absorption is one that is 10 to 25 mm, for example 15 mm.

The pH change due to the presence of gluconic acid is detected by a pH meter (for example by inserting the lateral flow device into a pH meter, or contacting the pH meter with the absorption pad of the lateral flow device or a solution released form the device) or pH paper.

### Microfluidic Devices

In one example, the solid support is a microfluidic device, which can be used to determine the presence and/or amount of one or more target agents in a sample, such as a liquid sample. Such devices are also referred to as “lab-on-a-chip” devices. The development of microfluidics and microfluidic techniques has provided improved chemical and biological research tools, including platforms for performing chemical reactions, combining and separating fluids, diluting samples, and generating gradients (for example, see U.S. Pat. No. 6,645,432).

A portable microfluidic device can be transported to almost any location. For microfluidic assays and devices, test samples (such as a liquid sample) can be supplied by an operator, for example using a micropipette. A test sample can be introduced into an inlet of a microfluidic system and the fluid may be drawn through the system by application of a vacuum source to the outlet end of the microfluidic system. Reagents may also be pumped in, for instance by using different syringe pumps filled with the required reagents. After one fluid is pumped into the microfluidic device, a second can be pumped in by disconnecting a line from the first pump and connecting a line from a second pump. Alternatively, valving may be used to switch from one pumped fluid to another. Different pumps can be used for each fluid to avoid cross contamination, for example when two fluids contain components that may react with each other or, when mixed, can affect the results of an assay or reaction. Continuous flow systems can use a series of two different fluids passing serially through a reaction channel. Fluids can be pumped into a channel in serial fashion by switching, through valving, the fluid source that is feeding the tube. The fluids constantly move through the system in sequence and are allowed to react in the channel.

Microfluidic devices for analyzing a target analyte are known, and can be adapted using the disclosed system to detect a target of interest. For example devices from Axis Shield (Scotland), such as the Afinion analyzer, analyzers from Claros (Woburn, Mass.), and devices from Advanced Liquid Logic (Morrisville, N.C.) such as those based on eletrowetting. Other exemplary devices are described in US Patent Publication Nos. 20110315229; 20100279310; 2012001830 and 2009031171.

In a particular example, the microfluidic device controls the movement of the sample and other liquids, dispenses reagents, and merges or splits a micro-size droplet in the microfluidic device via the voltage applied to the flow versus the device. The device can include a sample entry port, where the sample is introduced into the device. The device can also include an area containing buffer reagents, and area containing GOx (for example a nucleic acid-GOx conjugate which may be attached to a solid support, such as beads or graphene oxide), an area containing one or more enzyme substrates, such as glucose, a means to detect pH (e.g., pH meter or pH paper) or combinations thereof. The device includes one more mixing chambers, where desired reactions can occur.

In one example, the device includes a first chamber where target agent in the sample, if present, releases GOx from the solid support. The device also includes a region upstream of the first chamber, which can contain glucose or can be where the reaction of GOx converting glucose to gluconic acid occurs. In one example, the product from the first chamber (e.g., the GOx) passes thru the region containing the glucose, and enters the second chamber where gluconic acid is produced.

In one example, the device includes a first chamber containing FNA (which may be attached to a solid support) where target agent in the sample, if present, binds to a FNA, such as a DNAzyme, and releases a portion of the substrate strand of the FNA that can complete with binding of a nucleic acid-GOx conjugate on a solid support. The released portion of the substrate strand can enter a second chamber containing the nucleic acid-GOx conjugate on a solid support, where the released portion of the substrate strand competes with the nucleic acid-GOx conjugate, thereby releasing GOx from the solid support. The device also includes a region upstream of the second chamber, which can contain glucose or can be where the reaction of GOx converting glucose to gluconic acid occurs. In one example, the product from the second chamber (e.g., the GOx) passes thru the region containing the glucose, and enters the third chamber where gluconic acid is produced.

In one example, the device includes a first chamber containing a recognition molecule-GOx-target analogue complex (see FIG. 10A, which may be attached to a solid support), where target agent in the sample, if present, binds to the complex, and releases the GOx-target analogue portion of the complex. The device also includes a region upstream of the first chamber, which can contain glucose or can be where the reaction of GOx converting glucose to gluconic acid occurs. In one example, the product from the first chamber (e.g., the GOx) passes thru the region containing the glucose, and enters the second chamber where gluconic acid is produced.

In one example, the device includes a first chamber containing a first recognition molecule (see FIG. 10B), which may be attached to a solid support. The target in the sample, if present, binds to the first recognition molecule. The device also includes a second chamber connected to the first chamber, wherein the second chamber contains GOx conjugated to a second recognition molecule, which is allowed to interact with the first recognition molecule-target complex formed in the first chamber. This creates a first recognition molecule-target agent-second recognition molecule-GOx complex, for example in the first chamber. The device also includes a third chamber connected to the first chamber, wherein the third chamber contains glucose, which is allowed to interact with the first recognition molecule-target agent-second recognition molecule-GOx complex formed in the first chamber. This results in formation of gluconic acid, for example in the first chamber.

One skilled in the art will appreciate that other configuration as possible, for example more regions or mixing chambers if multiple targets are to be detected in the same sample on the same device. For example the device can have discrete regions and chambers for each target to be detected. In such an example, the microfluidic device may include multiple exit ports, one for each target. In another example, the device includes a first chamber where target agent in the sample, if present, binds to the recognition molecule present on the device, thereby creating a target agent-recognition molecule complex.

### Recognition Molecules that Specifically Bind the Target

The recognition molecule that specifically binds to the target agent, and thus permits detection of the target agent, can be a nucleic acid molecule (such as an FNA), protein, peptide nucleic acid, polymer, small organic molecule, an antibody, and the like. For example, the molecule that specifically binds to the target agent can be any substance that specifically binds to the target agent. In some examples, for example if the recognition molecule is a FNA, upon such binding, the molecule undergoes changes such as folding, binding, or releasing, which in some examples causes release of GOx conjugated to the molecule.

In one example the molecule that specifically binds to the target is an antibody (such as a monoclonal or polyclonal antibody or fragment thereof) or an antigen. Antibodies that are specific for a variety of target agents are commercially available, or can be generated using routine methods.

In one example the molecule that specifically binds to the target agent is protein that binds with high specificity to the target.

In yet another example, the recognition molecule that specifically binds to the target is a nucleic acid or other analogue, such as a peptide nucleic acid (PNA), locked nucleic acid (LNA), or any chemically modified nucleotide analogue. For example, the nucleic acid molecule can be composed of DNA or RNA, such as one that includes naturally occurring and/or modified bases. In an example when the target is a nucleic acid molecule (such as DNA or RNA) the recognition nucleic acid molecule can have a sequence that is complementary to the sequence of the target nucleic acid molecule, such that the target nucleic acid and recognition molecule can hybridize to one another. In one example, the nucleic acid molecule is a ribozyme which can detect a corresponding cofactor or target agent. A ribozyme is an RNA molecule with catalytic activity, for example RNA splicing activity. When ribozymes function, they often require a cofactor, such as metal ions (e.g., Mg²⁺) for their enzymatic activity. Such a cofactor can be the target agent detected based on ribozyme activity. Thus, as cofactors support ribozyme activity and ribozyme activity can be an indicator of the presence of the cofactor, or target agent.

Functional Nucleic Acids (FNAs)

FNAs are nucleic acid molecules (e.g., DNA or RNA) that can be used as enzymes (for catalysis), receptors (for binding to a target), or both. FNAs are known, and can be selected, to bind to a wide range of targets with high affinity and specificities. FNA sequences that can be modified or adapted to be used in the methods and sensors provided herein, are known in the art (e.g., see U.S. Pat. No. 8,058,415). One example of a FNA is a catalytic nucleic acid. The catalytic active nucleic acids can be catalytic DNA/RNA, also known as DNAzymes/RNAzymes, deoxyribozymes/ribozymes, DNA enzymes/RNA enzymes. Catalytic active nucleic acids can also contain modified nucleic acids. Aptazymes, RNAzymes, and DNAzymes become reactive upon binding an analyte by undergoing a chemical reaction (for example, cleaving a substrate strand of the FNA). In each instance, the outcome of the reactive polynucleotide becoming reactive is to cause disaggregation of the aggregate and the release of at least one oligonucleotide. Other example of a FNA is an aptamer, which undergoes a conformational change upon binding to the target. Aptamers become reactive upon binding an analyte by undergoing a conformational change.

Thus, in one example the recognition molecule that specifically binds to the target is a functional DNA (Liu et al, Chem. Rev. 2009, 109, 1948-1998). Functional DNAs, including DNAzymes and DNA aptamers, are known in the art for numerous targets. Such FNAs can be selected from pools of DNA (usually 2-25 kDa) with ˜10¹⁵ random sequences via a process known as in vitro selection or Systematic Evolution of Ligands by EXponential enrichment (SELEX). DNAzymes and aptamers exhibit specific catalytic activity and strong binding affinity, respectively, to various targets. The targets can range from metal ions and small organic molecules to biomolecules and even viruses or cells.

Methods of identifying a FNA that is specific for a particular target agent are routine in the art and have been described in several patents (all herein incorporated by reference). For example U.S. Pat. Nos. 7,192,708; 7,332,283; 7,485,419; 7,534,560; and 7,612,185, and US Patent Publication Nos. 20070037171 and 20060094026, describe methods of identifying functional DNA molecules that can bind to particular ions, such as lead and cobalt. In addition, specific examples are provided. Although some of the examples describe functional DNA molecules with fluorophores, such labels are not required for the sensors described herein.

Aptamers are single stranded (ss) nucleic acids (such as DNA or RNA) that recognize targets with high affinity and specificity, which undergo a conformational change in the presence of their target analyte. For example, the cocaine aptamer binds cocaine as its corresponding target. Thus, aptamers can be used as a recognition molecule. In vitro selection methods can be used to obtain aptamers for a wide range of target molecules with exceptionally high affinity, having dissociation constants as high as in the picomolar range (Brody and Gold, J. Biotechnol. 74: 5-13, 2000; Jayasena, Clin. Chem., 45:1628-1650, 1999; Wilson and Szostak, Annu. Rev. Biochem. 68: 611-647, 1999). For example, aptamers have been developed to recognize metal ions such as Zn(II) (Ciesiolka et al., RNA 1: 538-550, 1995) and Ni(II) (Hofmann et al., RNA, 3:1289-1300, 1997); nucleotides such as adenosine triphosphate (ATP) (Huizenga and Szostak, Biochemistry, 34:656-665, 1995); and guanine (Kiga et al., Nucleic Acids Research, 26:1755-60, 1998); co-factors such as NAD (Kiga et al., Nucleic Acids Research, 26:1755-60, 1998) and flavin (Lauhon and Szostak, J. Am. Chem. Soc., 117:1246-57, 1995); antibiotics such as viomycin (Wallis et al., Chem. Biol. 4: 357-366, 1997) and streptomycin (Wallace and Schroeder, RNA 4:112-123, 1998); proteins such as HIV reverse transcriptase (Chaloin et al., Nucleic Acids Research, 30:4001-8, 2002) and hepatitis C virus RNA-dependent RNA polymerase (Biroccio et al., J. Virol. 76:3688-96, 2002); toxins such as cholera whole toxin and staphylococcal enterotoxin B (Bruno and Kiel, BioTechniques, 32: pp. 178-180 and 182-183, 2002); and bacterial spores such as the anthrax (Bruno and Kiel, Biosensors & Bioelectronics, 14:457-464, 1999). Compared to antibodies, DNA/RNA based aptamers are easier to obtain and less expensive to produce because they are obtained in vitro in short time periods (days vs. months) and with limited cost. In addition, DNA/RNA aptamers can be denatured and renatured many times without losing their biorecognition ability.

DNA/RNAzymes typically contain a substrate strand with a RNA base, and a catalytic or enzyme domain that recognizes a target. In some examples a co-factor, such as a metal ion, catalyzes substrate cleavage. For example, the lead DNAzyme binds lead as its corresponding target. Thus, DNA/RNAzymes can be used as a recognition molecule. Aptazymes are the combination of aptamer and DNAzymes or ribozymes. Aptazymes work when the target binds to the aptamers which either triggers DNAzyme/ribozyme activities or inhibits DNAzyme/ribozyme activities. Thus, aptazymes can be used as a recognition molecule.

### Enzymes that can Change pH

Any enzyme that can convert a molecule (e.g., glucose) into an agent that will increase or decrease pH (e.g., gluconic acid), can be used in the sensors and methods provided herein. Although particular examples herein are provided using glucose oxidase (GOx), one skilled in the art will appreciate that other enzymes can be used, in combination with their appropriate substrate. Particular examples are shown in Table 2 below.

TABLE 2
Exemplary enzymes that can change pH

| | | Enzyme | Product that |
|---|---|---|---|
| Enzyme | Exemplary GenBank # | Substrate | Alters pH |
| GOx | Proteins: AGI04246.1; | Glucose | Gluconic acid |
| (EC 1.1.3.4) | AHC55209.1; NP_001011574.1; | | |
| | AAA32695.1 (such as aa 23-605 | | |
| | of this sequence) and | | |
| | AAF59929.2 | | |
| | Nucleic acids: J05242.1; | | |
| | KF741791.1; X56443.1 and | | |
| | NM_001011574.1 | | |
| Ureases | Proteins: NP_176922.1; | urea | ammonia |
| (EC 3.5.1.5) | NP_001236214.1; and | | |
| | AFZ10165.1 | | |
| | Nucleic Acids: NM_105422.3 | | |
| | and M65260.1 | | |
| Acetylcholinesterase | Proteins: ADD38982.1; | acetylcholine | acetic acid |
| | AGM37743.1; and AAC02779.1 | | |
| | Nucleic acids: AJ251640.1 and | | |
| | X03439.1 | | |
| Alkaline | Proteins: YP_004252858.1; | Phosphate | Phosphate or |
| Phosphatase | AEH43950.1; and ADR19525.1 | group | Phosphoric |
| | Nucleic acids: | substrate | acid |
| | NC_015681.1:67685..69199 and | | |
| | NC_015160.1:1964619..1966034 | | |
| Glutaminase | Proteins: YP_004239353.1; | Glutamine | ammonia |
| | AEH89104.1; and | | |
| | YP_003657266.1 | | |
| | Nucleic acids: FJ899679.1; | | |
| | XM_004348003.1; and | | |
| | XM_004339007.1 | | |
| Adenosine | Proteins: CDS65116.1; | Adenosine | ammonia |
| aminohydrolase | AHM73938.1; and | | |
| | WP_023471170.1 | | |
| | Nucleic Acids: | | |
| | LK931482.1:1648110..1649111 | | |
| | and | | |
| | CP007448.1:2355010..2356008 | | |

To apply these enzymes in the sensors described herein, the GOx described in the examples herein can be replaced by one of these enzymes and the glucose replaced by the corresponding enzyme substrates listed above.

Although exemplary GENBANK® numbers are listed herein, the disclosure is not limited to the use of these sequences. Many other enzyme sequences are publicly available, and can thus be readily used in the disclosed methods. In one example, an enzyme having at least 70%, at least 75%, at least 80%, at least 85%, at least 90%, at least 95%, at least 98%, or at least 100% sequence identity to any of the GENBANK® numbers are listed herein that retains the ability to catalyze the conversion of an enzyme substrate into a product that increases or decreases pH of a solution, is used in the sensors disclosed herein. In addition, such enzymes that can be used with the disclosed sensors and methods are available from commercial sources, such as Sigma-Aldrich (St. Louis, Mo.).

### Attachment of Molecules

Methods of conjugating or attaching one agent to another agent are known in the art, and the disclosure is not limited to particular attachment methods. For example, a recognition molecule that can specifically bind to the target (such as an antibody, polymer, protein, FNA, or nucleic acid) can be attached to GOx and/or to a solid support (such as a conjugation pad, bead, graphene oxide, or multiwell plate) using conventional methods. Similarly, nucleic acid molecules used in the present disclosure, such as anchoring nucleic acids and nucleic acid-GOx conjugates, can be attached to a solid support (such as a conjugation pad, bead, graphene oxide, or multiwell plate) using conventional methods. The conjugation method used can be any chemistry that can covalently or non-covalently incorporate one molecule with another molecule. In some examples, a molecule (such as a recognition molecule-target analogue-GOx complex) is attached to a solid support, such as a conjugation pad of a lateral flow device, simply by suspending the molecule to be attached in a solution, applying the solution to the pad, and allowing the solution to dry.

In one example the method uses a reaction that forms covalent bonds including but not limited to those between amines and isothiocyanates, between amines and esters, between amines and carboxyls, between thiols and maleimides, between thiols and thiols, between azides and alkynes, and between azides and nitriles. In one example, the methods uses a reaction that forms bonds between streptavidin and biotin. In another example, the method uses a reaction that forms non covalent interactions including but not limited to those between antibodies and antigens, between FNAs and corresponding targets, and between organic chelators and metal ions.

In one example, the GOx is labeled with biotin thru covalent bonds including but not limited to those Biotin Labeling Kits (e.g., Sulfo-NHS-Biotin), and conjugate with biotinylated DNA using streptavidin or avidin as a linker. The immobilization method of GOx-DNA conjugate to beads includes but not limited to DNA hybridization, biotin-streptavidin interaction, and other covalent or non-covalent linkers.

In a specific example, GOx is conjugated to DNA by maleimide-thiol or isothiocyanate-amine reaction; then, the DNA-GOx conjugate is immobilized to magnetic beads via DNA hybridization with anchoring or FNA on the beads.

### Exemplary Targets/Analytes

The disclosed sensors can be designed to detect any target molecule agent of interest. Thus, the methods and devices provided herein can be used to detect any target agent of interest, such as the specific examples provided herein. As described above, selecting an appropriate recognition molecule that permits detection of the target agent, allows one to develop a sensor and a method that can be used to detect a particular target agent. Exemplary target agents are provided below; however one skilled in the art will appreciate that other target agents can be detected with the disclosed sensors and devices (such as the lateral flow devices and microfluidic devices provided herein) using the disclosed methods. In one example, the target is any agent that can specifically bind to a particular recognition molecule, such as an antibody, functional nucleic acid (e.g., DNAzyme or aptamer) or nucleic acid. Commercially available antibodies are available for numerous agents, such as proteins (e.g., cytokines, tumor antigens, etc.), metals, small organic compounds and nucleic acid molecules. In addition, methods of making antibodies, functional nucleic acids, and nucleic acid molecules that are specific for a particular target are well known in the art.

### Metals

In one example the target agent is a metal (e.g., elements, compounds, or alloys that have high electrical conductivity), such as a heavy metal or a nutritional metal. Metals occupy the bulk of the periodic table, while non-metallic elements can only be found on the right-hand-side of the Periodic Table of the Elements. A diagonal line drawn from boron (B) to polonium (Po) separates the metals from the nonmetals. Most elements on this line are metalloids, sometimes called semiconductors. Elements to the lower left of this division line are called metals, while elements to the upper right of the division line are called non-metals.

Target heavy metals include any metallic chemical element that has a relatively high density and is toxic, highly toxic or poisonous at low concentrations. Examples of target heavy metals include mercury (Hg), cadmium (Cd), arsenic (As), chromium (Cr), thallium (Tl), uranium (U), plutonium (Pu), and lead (Pb).

Target nutritional metal ions include those important in animal nutrition and may be necessary for particular biological functions, include calcium, iron, cobalt, magnesium, manganese, molybdenum, zinc, cadmium, sodium, potassium, lithium, and copper.

Antibodies specific for particular metals are known in the art. For example, Zhu et al. describe mAbs specific for chelated cadmium ions (J. Agric. Food Chem. 55:7648-53, 2007), Wylie et al. describe mAbs specific for mercuric ions (PNAS 89:4104-8, 1992), and Love et al. describe mAbs specific for inidium (Biochem. 32:10950-9, 1993). In addition, bifunctional derivatives of metal ion chelators (EDTA, DTPA, DOTA) can be covalently conjugated to proteins and loaded with the desired metal ion. These conjugates can be used to prepare hybridoma cell lines which synthesize metal-specific monoclonal antibodies. In addition, aptamers have been developed to recognize metal ions such as Zn(II) (Ciesiolka et al., RNA 1: 538-550, 1995) and Ni(II) (Hofmann et al., RNA, 3:1289-1300, 1997). Furthermore, DNAzymes specific for particular metal ions are known, such as lead, copper, uranium, zinc, mercury, cadmium and magnesium. Exemplary non-limiting structures that can be used in the disclosed sensors and methods for detecting metals are provided herein. One skilled in the art will appreciate that any known functional nucleic acid can be manipulated using the methods herein to detect a metal of interest.

### Pathogens/Microbes

Any pathogen or microbe can be detected using the sensors and methods provided herein. For example, particular antimicrobial antigens and nucleic acid molecules (such as DNA or RNA), as well as bacterial spores, can be detected. In some examples, a particular microbial cell is detected, or a particular virus. In some examples, intact microbes are detected, for example by detecting a target surface protein (such as a receptor) using sensors that include for example antibodies, DNAzymes, or DNA aptamers specific for the target protein. For example, antibodies that can be used with the disclosed sensors are available from commercial sources, such as Novus Biologicals (Littleton, Colo.) and ProSci Incorporated (Poway, Calif.) provide E. coli-specific antibodies; KPL (Gaithersburg, Md.) provides Listeria-specific antibodies; Thermo Scientific/Pierce Antibodies (Rockford, Ill.) provides antibodies specific for several microbes, including bacteria and viruses, such as influenza A, HIV-1, HSV 1 and 2, E. coli, Staphylococcus aureus, Bacillus anthracis and spores thereof, Plasmodium, and Cryptosporidium. In addition, aptamers specific for microbial proteins can be used with the disclosed sensors, such as those specific for HIV reverse transcriptase (Chaloin et al., Nucleic Acids Research, 30:4001-8, 2002) and hepatitis C virus RNA-dependent RNA polymerase (Biroccio et al., J. Virol. 76:3688-96, 2002); toxins such as cholera whole toxin and staphylococcal enterotoxin B (Bruno and Kiel, BioTechniques, 32: pp. 178-180 and 182-183, 2002); and bacterial spores such as anthrax (Bruno and Kiel, Biosensors & Bioelectronics, 14:457-464, 1999). In addition, DNAzymes specific for bacteria can be used with the disclosed sensors, such as those specific for Escherichia coli-K12 (Ali et al., Angewandte Chemie International Edition. 50, 3751-4, 2011; Li, Future Microbiol. 6, 973-976, 2011; and Aguirre, et al., J. Visualized Experiments. 63, 3961, 2012). In other examples, a conserved DNA or RNA specific to a target microbe is detected, for example by obtaining nucleic acids from a sample (such as from a sample known or suspected of containing the microbe), wherein the resulting nucleic acids (such as DNA or RNA or both) are then contacted with the sensors disclosed herein (which include the complementary nucleic acid sequence that can hybridize to the target nucleic acid). One skilled in the art will appreciate that any known functional nucleic acid can be manipulated using the methods herein to detect a pathogen of interest.

Exemplary pathogens include, but are not limited to, viruses, bacteria, fungi, nematodes, and protozoa. A non-limiting list of pathogens that can be detected using the sensors and methods provided herein are provided below.

For example, target viruses include positive-strand RNA viruses and negative-strand RNA viruses. Exemplary target positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain). Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses).

Viruses also include DNA viruses. Target DNA viruses include, but are not limited to: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B 19).

Another group of viruses includes Retroviruses. Examples of target retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In one example, the virus detected with the disclosed methods or sensors is one or more of the following: HIV-1 (for example an HIV antibody, p24 antigen, or HIV genome); Hepatitis A virus (for example an Hepatitis A antibody, or Hepatitis A viral genome); Hepatitis B (HB) virus (for example an HB core antibody, HB surface antibody, HB surface antigen, or HB viral genome); Hepatitis C (HC) virus (for example an HC antibody, or HC viral genome); Hepatitis D (HD) virus (for example an HD antibody, or HD viral genome); Hepatitis E virus (for example a Hepatitis E antibody, or HE viral genome); a respiratory virus (such as influenza A & B, respiratory syncytial virus, human parainfluenza virus, or human metapneumovirus), or West Nile Virus.

In one example, the sensors and methods provided herein can distinguish between an infectious versus a non-infectious virus.

Pathogens also include bacteria. Bacteria can be classified as gram-negative or gram-positive. Exemplary target gram-negative bacteria include, but are not limited to: Escherichia coli (e.g., K-12 and 0157:H7), Shigella dysenteriae, and Vibrio cholerae. Exemplary target gram-positive bacteria include, but are not limited to: Bacillus anthracis, Staphylococcus aureus, Listeria, pneumococcus, gonococcus, and streptococcal meningitis. In one example, the bacteria detected with the disclosed methods and sensors is one or more of the following: Group A Streptococcus; Group B Streptococcus; Helicobacter pylori; Methicillin-resistant Staphylococcus aureus; vancomycin-resistant enterococci; Clostridium difficile; E. coli (e.g., Shiga toxin producing strains); Listeria; Salmonella; Campylobacter; B. anthracis (such as spores); Chlamydia trachomatis; and Neisseria gonorrhoeae.

Protozoa, nemotodes, and fungi are also types of pathogens. Exemplary target protozoa include, but are not limited to, Plasmodium (e.g., Plasmodium falciparum to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, and Toxoplasma. Exemplary target fungi include, but are not limited to, Coccidiodes immitis and Blastomyces dermatitidis.

In one example, bacterial spores are detected. For example, the genus of Bacillus and Clostridium bacteria produce spores that can be detected. Thus, C. botulinum, C. perfringens, B. cereus, and B. anthracis spores can be detected (for example detecting anthrax spores). One will also recognize that spores from green plants can also be detected using the methods and devices provided herein.

### Proteins

The disclosed sensors and methods also permit detection of a variety of proteins, such as cell surface receptors, cytokines, antibodies, hormones, as well as toxins. In particular examples, the recognition molecule that can specifically bind to a protein target is a protein (such as an antibody) or nucleic acid (such as a functional nucleic acid). In some examples, a target protein is selected that is associated with a disease or condition, such that detection (or absence) of the target protein can be used to infer information (such as diagnostic or prognostic information for the subject from whom the sample is obtained) relating to the disease or condition. Antibodies specific for particular proteins are known in the art. For example, such antibodies are available from commercial sources, such as Invitrogen, Santa Cruz Biotechnology (Santa Cruz, Calif.); ABCam (Cambridge, Mass.) and IBL International (Hamburg, Germany). Exemplary non-limiting structures that can be used in the disclosed sensors and methods for detecting proteins are provided herein. One skilled in the art will appreciate that any known functional nucleic acid can be manipulated using the methods herein to detect a protein of interest.

In one example the protein is a cytokine. Cytokines are small proteins secreted by immune cells that have effects on other cells. Examples of target cytokines include interleukins (IL) and interferons (IFN), and chemokines, such as IL-1, IL-2, IL-4, IL-6, IL-8, IL-10, IFN-γ, IFN-β, transforming growth factor (TGF-β), and tumor necrosis factor (TNF)-α.

In one example the protein is a hormone. A hormone is a chemical messenger that transports a signal from one cell to another. Examples of target hormones include plant and animal hormones, such as endocrine hormones or exocrine hormones. Particular examples include follicle stimulating hormone (FSH), human chorionic gonadotropin (hCG), thyroid stimulating hormone (TSH), growth hormone, progesterone, and the like.

In yet another example the protein is a toxin. Toxins are poisonous substances produced by cells or organisms, such as plants, animals, microorganisms (including, but not limited to, bacteria, viruses, fungi, rickettsiae or protozoa). Particular examples of target toxins include botulinum toxin, ricin, diphtheria toxin, Shiga toxin, Cholera toxin, Staphylococcal enterotoxin B, and anthrax toxin. In another example, the toxin is an environmental toxin. In one example the toxin is a mycotoxin, such as: aflatoxin, citrinin, ergot alkaloids, patulin, fusarium toxins, or ochratoxin A. In one example the target toxin is a cyanotoxin, such as: microcystins, nodularins, anatoxin-a, aplysiatoxins, cylindrospermopsins, lyngbyatoxin-a, and saxitoxins. In one example the target toxin is an endotoxin, hemotoxin, necrotoxin, neurotoxin, or cytotoxin.

In one example, the target protein is a tumor-associated or tumor-specific antigen, such as CA-125 (ovarian cancer marker), alphafetoprotein (AFP, liver cancer marker); carcinoembryonic antigen (CEA; bowel cancers), BRCA1 and 2 (breast cancer), and the like.

In one example the target protein is a fertility-related biomarker, such as hCG, luteinizing hormone (LH), follicle-stimulating hormone (FSH), or fetal fibrinogen.

In one example the target protein is a diagnostic protein, such as prostate-specific antigen (PSA, for example GenBank® Accession No. NP_{—}001025218), C reactive protein, cyclic citrullinate peptides (CCP, for example to diagnose rheumatoid arthritis) or glycated hemoglobin (HbA1c). In another example, the protein is one found on the surface of a target microbe or cell, such as a bacterial cell, virus, spore, or tumor cell. Such proteins, such as receptors, may be specific for the microbe or cell (for example HER2, IGF1R, EGFR or other tumor-specific receptor noted below in “nucleic acids”). In on example the protein is prostate-specific antigen (PSA, for example GenBank® Accession No. NP_{—}001025218), which can be detected using an antibody or PSA-specific aptamer (e.g., see Savory et al., Biosensors & Bioelectronics 15:1386-91, 2010 and Jeong et al., Biotechnology Letters 32:378-85, 2010).

### Nucleic Acids

The disclosed sensors and methods also permit detection of nucleic acid molecules, such DNA and RNA, such as a DNA or RNA sequence that is specific for a particular pathogen or cell of interest. For example, target pathogens can have conserved DNA or RNA sequences specific to that pathogen (for example conserved sequences are known in the art for HIV, bird flu and swine flu), and target cells may have specific DNA or RNA sequences unique to that cell, or provide a way to distinguish a target cell from another cell (such as distinguish a tumor cell from a benign cell).

In some examples, a target sequence is selected that is associated with a disease or condition, such that detection of hybridization between the target nucleic acid and a sensor provided herein can be used to infer information (such as diagnostic or prognostic information for the subject from whom the sample is obtained) relating to the disease or condition.

In specific non-limiting examples, the target nucleic acid sequence is associated with a tumor (for example, a cancer). Numerous chromosome abnormalities (including translocations and other rearrangements, reduplication (amplification) or deletion) have been identified in neoplastic cells, especially in cancer cells, such as B cell and T cell leukemias, lymphomas, breast cancer, ovarian cancer, colon cancer, neurological cancers and the like.

Exemplary target nucleic acids include, but are not limited to: the SYT gene located in the breakpoint region of chromosome 18q11.2 (common among synovial sarcoma soft tissue tumors); HER2, also known as c-erbB2 or HER2/neu (a representative human HER2 genomic sequence is provided at GENBANK® Accession No. NC_{—}000017, nucleotides 35097919-35138441) (HER2 is amplified in human breast, ovarian, gastric, and other cancers); p16 (including D9S1749, D9S1747, p16(INK4A), p14(ARF), D9S1748, p15(INK4B), and D9S1752) (deleted in certain bladder cancers); EGFR (7p12; e.g., GENBANK® Accession No. NC_{—}000007, nucleotides 55054219-55242525), MET (7q31; e.g., GENBANK® Accession No. NC_{—}000007, nucleotides 116099695-116225676), C-MYC (8q24.21; e.g., GENBANK® Accession No. NC_{—}000008, nucleotides 128817498-128822856), IGF1R (15q26.3; e.g., GENBANK® Accession No. NC_{—}000015, nucleotides 97010284-97325282), D5S271 (5p15.2), KRAS (12p12.1; e.g. GENBANK® Accession No. NC_{—}000012, complement, nucleotides 25249447-25295121), TYMS (18p11.32; e.g., GENBANK™ Accession No. NC_{—}000018, nucleotides 647651-663492), CDK4 (12q14; e.g., GENBANK® Accession No. NC_{—}000012, nucleotides 58142003-58146164, complement), CCND1 (11q13, GENBANK® Accession No. NC_{—}000011, nucleotides 69455873-69469242), MYB (6q22-q23, GENBANK® Accession No. NC_{—}000006, nucleotides 135502453-135540311), lipoprotein lipase (LPL) (8p22; e.g., GENBANK® Accession No. NC_{—}000008, nucleotides 19840862-19869050), RB1 (13q14; e.g., GENBANK® Accession No. NC_{—}000013, nucleotides 47775884-47954027), p53 (17p13.1; e.g., GENBANK® Accession No. NC_{—}000017, complement, nucleotides 7512445-7531642), N-MYC (2p24; e.g., GENBANK® Accession No. NC_{—}000002, complement, nucleotides 15998134-16004580), CHOP (12q13; e.g., GENBANK® Accession No. NC_{—}000012, complement, nucleotides 56196638-56200567), FUS (16p11.2; e.g., GENBANK® Accession No. NC_{—}000016, nucleotides 31098954-31110601), FKHR (13p14; e.g., GENBANK® Accession No. NC_{—}000013, complement, nucleotides 40027817-40138734), aALK (2p23; e.g., GENBANK® Accession No. NC_{—}000002, complement, nucleotides 29269144-29997936), Ig heavy chain, CCND1 (11q13; e.g., GENBANK® Accession No. NC_{—}000011, nucleotides 69165054-69178423), BCL2 (18q21.3; e.g., GENBANK® Accession No. NC_{—}000018, complement, nucleotides 58941559-59137593), BCL6 (3q27; e.g., GENBANK® Accession No. NC_{—}000003, complement, nucleotides 188921859-188946169), AP1 (1p32-p31; e.g., GENBANK® Accession No. NC_{—}000001, complement, nucleotides 59019051-59022373), TOP2A (17q21-q22; e.g., GENBANK® Accession No. NC_{—}000017, complement, nucleotides 35798321-35827695), TMPRSS (21q22.3; e.g., GENBANK® Accession No. NC_{—}000021, complement, nucleotides 41758351-41801948), ERG (21q22.3; e.g., GENBANK® Accession No. NC_{—}000021, complement, nucleotides 38675671-38955488); ETV1 (7p21.3; e.g., GENBANK® Accession No. NC_{—}000007, complement, nucleotides 13897379-13995289), EWS (22q12.2; e.g., GENBANK™ Accession No. NC_{—}000022, nucleotides 27994017-28026515); FLI1 (11q24.1-q24.3; e.g., GENBANK® Accession No. NC_{—}000011, nucleotides 128069199-128187521), PAX3 (2q35-q37; e.g., GENBANK® Accession No. NC_{—}000002, complement, nucleotides 222772851-222871944), PAX7 (1p36.2-p36.12; e.g., GENBANK® Accession No. NC_{—}000001, nucleotides 18830087-18935219), PTEN (10q23.3; e.g., GENBANK® Accession No. NC_{—}000010, nucleotides 89613175-89718512), AKT2 (19q13.1-q13.2; e.g., GENBANK® Accession No. NC_{—}000019, complement, nucleotides 45428064-45483105), MYCL1 (1p34.2; e.g., GENBANK™ Accession No. NC_{—}000001, complement, nucleotides 40133685-40140274), REL (2p13-p12; e.g., GENBANK® Accession No. NC_{—}000002, nucleotides 60962256-61003682) and CSF1R (5q33-q35; e.g., GENBANK® Accession No. NC_{—}000005, complement, nucleotides 149413051-149473128).

In examples where the target molecule is a nucleic acid molecule, the sample to be tested can be treated with agents that permit disruption of the cells or pathogen. The nucleic acid molecules can be extracted or isolated, and then exposed to a sensor disclosed herein, such as a sensor that includes nucleic acid-GOx conjugates and a nucleic acid molecule as the recognition molecule having a sequence that is complementary to the target DNA or RNA sequence, such that the complementary nucleic acid sequence can hybridize to the target nucleic acid, thereby permitting detection of the target nucleic acid.

### Recreational and Other Drugs

The disclosed sensors and methods also permit detection of a variety of drugs, such as pharmaceutical or recreational drugs. Antibodies specific for particular drugs are known in the art. For example, antibodies to tetrahydrocannabinol, heroin, cocaine, caffeine, and methamphetamine are available from AbCam (Cambridge, Mass.). In particular examples, the recognition molecule that can specifically bind to the drug target is a nucleic acid (such as a functional nucleic acid, such as an aptamer or DNAzyme). Exemplary non-limiting structures that can be used in the disclosed sensors and methods for detecting drugs are provided herein. One skilled in the art will appreciate that any known functional nucleic acid can be manipulated using the methods herein to detect a drug of interest.

For example, the presence of caffeine, cocaine, opiates and opioids (such as oxycodone), cannabis (for example by detecting tetrahydrocannabinol (THC)), heroin, methamphetamines, crack, ethanol, acetaminophen, benzodiazepines, methadone, phencyclidine, or tobacco (for example by detecting nicotine), can be detected using the disclosed sensors and methods. In one example, the target is a therapeutic drug, such as theophylline, methotrexate, tobramycin, cyclosporine, rapamycin, or chloramphenicol.

Cells

The disclosed sensors and methods also permit detection of a variety of cells, such as tumor or cancer cells, as well as other diseased cells. In on example, the sensor can distinguish between a tumor cell and a normal cell of the same cell type, such as a normal breast cell from a cancerous breast cell. Tumors are abnormal growths which can be either malignant or benign, solid or liquid (for example, hematogenous). In some examples, cells are detected by using a sensor that includes a recognition molecule specific for a surface protein, such as a receptor on the surface of the cell. For example, antibodies specific for particular cells are known in the art. Usually, such antibodies recognize a surface protein expressed by the cell, such as a receptor. For example, such antibodies are available from commercial sources, such as AbCam and Santa Cruz Biotechnology. In other examples, cells are detected by using a sensor that includes a recognition molecule specific for a nucleic acid found in the tumor cell.

Examples of target hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (including low-, intermediate-, and high-grade), multiple myeloma, Waldenström's macroglobulinemia, heavy chain disease, myelodysplastic syndrome, mantle cell lymphoma and myelodysplasia.

Examples of target solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Thus, in some examples the sensors and devices provided herein permit detection of such tumor cells using the disclosed methods.

### Kits

The disclosure also provides kits that include one or more of the sensors disclosed herein, for example sensors that are part of a lateral flow device or a microfluidic device. For example, a kit can include at least 2 different sensors permitting detection of at least two different target agents, such as at least 3, at least 4, at least 5, or at least 10 different sensors. In a specific example, a kit can include at least 2 different lateral flow devices or microfluidic devices permitting detection of at least two different target agents, such as at least 3, at least 4, at least 5, or at least 10 different lateral flow devices or microfluidic devices.

The kits can contain the sensor, lateral flow device, or microfluidic device, and a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, wherein the packaging unit may have compartments into which one or more components of the kit can be placed. In other examples, a kit includes one or more containers, for instance vials, tubes, and the like that can retain, for example, one or more biological samples to be tested, positive and/or negative control samples or solutions (such as, a positive control sample containing the target), diluents (such as, phosphate buffers, or saline buffers), a pH meter, pH paper, and/or wash solutions (such as, Tris buffers, saline buffer, distilled water, or any of the buffers listed in Example 1).

Kits can include other components, such as a buffer, a chart for correlating a detected pH or color change on pH paper and amount of target agent present, glucose, glucose oxdiase, or combinations thereof. For example, the kit can include a vial containing one or more of the sensors disclosed herein and a separate vial containing glucose.

Other kit embodiments include syringes, finger-prick devices, alcohol swabs, gauze squares, cotton balls, bandages, latex gloves, incubation trays with variable numbers of troughs, adhesive plate sealers, data reporting sheets, which may be useful for handling, collecting and/or processing a biological sample. Kits may also optionally contain implements useful for introducing samples onto a lateral flow device or a microfluidic device, including, for example, droppers, Dispo-pipettes, capillary tubes, rubber bulbs (e.g., for capillary tubes), and the like. Still other kit embodiments may include disposal means for discarding a used device and/or other items used with the device (such as patient samples, etc.). Such disposal means can include, without limitation, containers that are capable of containing leakage from discarded materials, such as plastic, metal or other impermeable bags, boxes or containers.

In some examples, a kit will include instructions for the use of a sensor, microfluidic device, or lateral flow device. The instructions may provide direction on how to apply sample to the sensor or device, the amount of time necessary or advisable to wait for results to develop, and details on how to read and interpret the results of the test. Such instructions may also include standards, such as standard tables, graphs, or pictures for comparison of the results of a test. These standards may optionally include the information necessary to quantify target analyte using the sensor or device, such as a standard curve relating the pH detected to an amount of target present in the sample.

### Example 1

### Materials

This example describes the materials used in Examples 2-10 below.

Streptavidin-coated magnetic beads (1 μm) and Amicon centrifugal filters were from Bangs Laboratories Inc. (Fishers, Ind.) and Millipore Inc. (Billerica, Mass.), respectively. Glucose oxidase (GOx) type VII (>100 units/mg) from Aspergillus niger, sulfosuccinimidyl-4-(N-maleimidomethyl)cyclohexane-1-carboxylate (sulfo-SMCC), Tris(2-carboxyethyl)phosphine hydrochloride (TCEP), metal salts, glucose, graphene oxide stock solution (2 mg/mL in water), Ricin B chain from Ricinus communis (castor bean), and other chemicals for buffers and solvents were from Sigma-Aldrich, Inc. (St. Louis, Mo.). 2% milk was purchased from a local store. Ricin B chain was spiked into milk at levels of 4, 8, 16, 32, 50, 70 and 100 μg/mL, and used as stock solution.

The following oligonucleotides (from left to right: 5′ to 3′) were obtained from Integrated DNA Technologies, Inc. (Coralville, Iowa):

Biotin-DNA for DNA-GOx immobilization onto
magnetic beads for Pb²⁺ detection:
(SEQ ID NO: 1)
Biotin-AAAAAAAAAAAATATAGTGTAGCATCGGACA
DNA for DNA-GOx conjugation for Pb²⁺ detection:
(SEQ ID NO: 2)
TGTCCGATGCTAAAAAAAAAAAAA-SH
Pb²⁺-dependent DNAzyme:
(SEQ ID NO: 3)
ACAGACATCTCTTCTCCGAGCCGGTCGAAATAGTGTAG
Substrate for Pb²⁺-dependent DNAzyme
(SEQ ID NO: 4)
TGTCCGATGCTACACTATrAGGAAGAGATGTCTGT

The substrate and DNAzyme complex were annealed at 85° C. for 15 min in buffers before use.

Biotin-DNA for DNA-GOx immobilization onto
magnetic beads for cocaine detection:
(SEQ ID NO: 5)
TCACAGATGAGTAAAAAAAAAAAA-biotin
DNA for DNA-GOx conjugation for cocaine
detection:
(SEQ ID NO: 6)
HS-AAAAAAAAAAAAGTCTCCCGAGAT
DNA for DNA-GOx conjugation for ricin detection
(the ricin aptamer with 12 “A”s as a DNA base
spacer for conjugation)
(SEQ ID NO: 7)
HS- AAAAAAAAAAAA
ACACCCACCGCAGGCAGACGCAACGCCTCGGAGACTAGCC
Cocaine aptamer (Coc-Apt):
(SEQ ID NO: 8)
TTTTTTACTCATCTGTGAATCTCGGGAGACAAGGATAAATCCTTCAAT
GAAGTGGGTCTCCC

Buffers used:

Buffer A (for DNA-GOx conjugation): 100 mM sodium phosphate buffer, pH 7.3, 0.1 M NaCl

Buffer B (for DNA-GOx immobilization and DNA invasion): 5 mM sodium phosphate buffer, pH 7.2, 200 mM NaCl, 0.05% Tween-20

Buffer C (for Pb²⁺-dependent DNAzyme): 5 mM HEPES, pH 7.1, 200 mM NaCl

Buffer D (for cocaine aptamer): 10 mM HEPES, pH 7.3, 200 mM NaCl, 0.05% Tween-20

### Example 2

### Procedure for DNA-GOx Conjugation

This example describes methods of generating a DNA-GOx conjugate. One skilled in the art will appreciate that the sequences used for lead, cocaine, or ricin detection described in Example 1 (e.g., SEQ ID NOS: 2, 6, and 7, respectively) can be modified to work with any DNAzyme or aptamer of interest.

The conjugation method was previously published (FIG. 14).⁶ To 60 μL of 1 mM thiol-DNA in Millipore water, 5 μL of 1 M sodium phosphate buffer at pH 5.5 and 5 μL of 30 mM TCEP in Millipore water were added and mixed. This mixture was kept at room temperature for 2 hours and then purified by Amicon-10K using Buffer A by 8 times. For GOx conjugation, 400 μL of 20 mg/mL GOx in Buffer A was mixed with 2 mg of sulfo-SMCC. After vortexing, the solution was placed on a shaker for 2 hour at room temperature. The mixture was then centrifuged and the insoluble excess sulfo-SMCC was removed. The clear solution was then purified by Amicon-100K using Buffer A by 8 times. The purified solution of sulfo-SMCC-activated GOx was mixed with the above solution of thiol-DNA (SEQ ID NO: 2, 6, or 7). The resulting solution was kept at room temperature for 48 hours. To remove un-reacted thiol-DNA, the solution was purified by Amicon-100K 8 times using Buffer A.

### Example 3

### Immobilization of DNA-GOx to Magnetic Beads

This example describes methods of immobilizing the DNA-GOx conjugates generated in Example 2 for lead and cocaine to magnetic beads. One skilled in the art will appreciate that other supports can be used in place of the magnetic beads, such as a membrane, glass substrate, or other type of bead, such as a gold bead, and methods of immobilizing to such surfaces is well known in the art. In addition, one skilled in the art will understand that the specific DNA sequences used for detecting lead or cocaine (which are at least partially based on the sequence of the DNAzyme or aptamer specific for the target, can be modified for any target of interest.

To prepare DNA-GOx immobilized MBs for Pb²⁺ detection, a portion of 1 mL 1 mg/mL solution of streptavidin-coated magnetic beads (MBs) in a microtube was placed close to a magnetic rack for 1 minute. The clear solution was discarded and replaced by 1 mL of Buffer B. This buffer exchange procedure was repeated twice. Then, 12 μL 0.5 mM biotin-DNA (SEQ ID NO: 1 for lead) in water was added to the 1 mL MB solution and mixed on a roller for 30 minutes at room temperature. After that, the MBs were washed twice using Buffer B to remove excess biotin-DNA. Later, 12 μL of 0.5 mM DNA-GOx generated in Example 2 (about 20 mg/mL total GOx) conjugate in Buffer B was added to the solution and well mixed at room temperature for 30 minutes. Excess DNA-GOx conjugate was washed off by Buffer B for five times and was recycled for further use by condensing the washing solutions using an Amicon-100K. The MBs residue from 100 μL MB solution after removal of solvent was used for each test.

To prepare DNA-GOx immobilized MBs for cocaine detection, all the procedures were the same except for adding 12 μL 0.5 mM biotin-DNA (SEQ ID NO: 5) and 12 μL 0.5 mM cocaine aptamer (SEQ ID NO: 8) instead of adding only 12 μL 0.5 mM biotin-DNA for Pb²⁺ detection above. The MBs residue from 40 μL MB solution after removal of solvent was used for each test.

### Example 4

### Immobilization of DNA-GOx to Graphene Oxide

This example describes methods of immobilizing the DNA-GOx conjugate generated in Example 2 for ricin to graphene oxide. One skilled in the art will appreciate that other supports can be used in place of the graphene oxide, such as a membrane, carbon sphere/sheets, glass substrate, or a bead, such as a gold or metallic bead, and methods of immobilizing to such surfaces is well known in the art. In addition, one skilled in the art will understand that the specific DNA sequences used for detecting ricin (e.g., SEQ ID NO: 7), can be modified for any target of interest.

To prepare DNA-GOx immobilized graphene oxide for ricin detection, 12 μL of 0.5 mM DNA-GOx conjugate generated in Example 2 (about 20 mg/mL total GOx) in Buffer B was added to 300 μL buffer A containing a certain concentration of GO (about 20 μg/mL). The mixture was placed on a shaker for 1 hour at room temperature. Afterwards, the DNA-GOx-GO was washed twice using buffer D to remove excess GOx-DNA, and then dispersed in 600 μL buffer D.

### Example 5

### Pb²⁺ Detection by pH Meter

This example describes the use of a lead DNAzyme and magnetic beads containing DNA-GOx as generally illustrated in FIGS. 4A and 4B. One skilled in the art will appreciate that similar methods can be used to for other targets and DNAzymes.

Because portable pH meters are used by users to monitor the pH of water, soil and other environmental samples, enabling portable pH meters to detect environmental pollutants such as lead (Pb²⁺) can help the users to detect many analytes related to environment with only a single meter. One skilled in the art will appreciate that pH paper can be used instead of a meter, for example for qualitative or semi-quantitative detection.

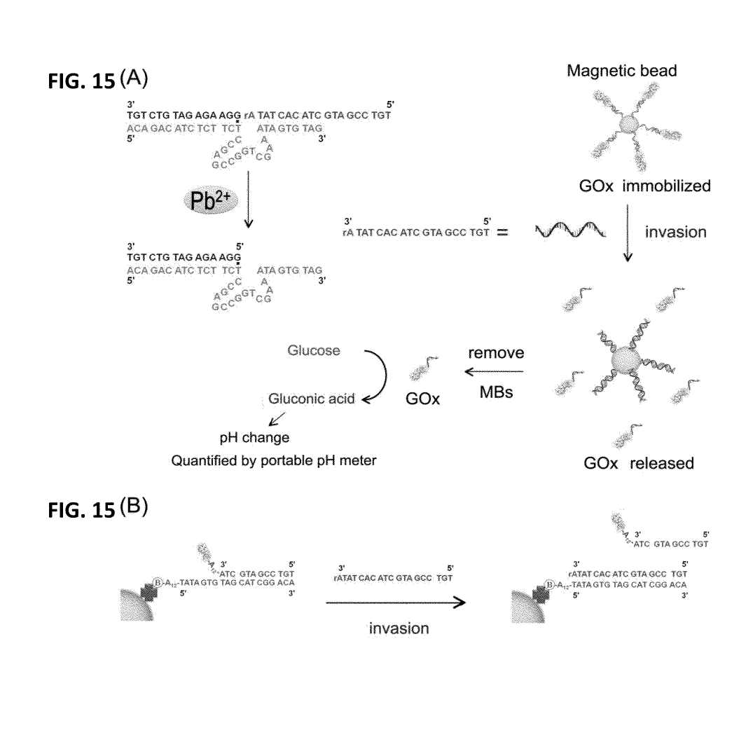

Due to the possibility that heavy metal ions such as Pb²⁺ may bind to GOx and vary its activity, a DNA invasive approach shown in FIGS. 15A and 4B was used. First, the DNA duplex containing a Pb²⁺-dependent DNAzyme⁸⁻¹⁰ (green, bottom strand) and its cleavable substrate (top strand) underwent cleavage when mixing with samples containing Pb²⁺ (FIG. 15A). The cleaved ssDNA (red, top right piece) dissociated from the DNA duplex and could compete with the DNA-GOx conjugates immobilized on magnetic beads (MBs) through DNA hybridization (FIG. 15B). As a result, the DNA-GOx conjugates were released from the surface of MBs into solution because of the shorter complementary DNA strand. After removal of MBs by a magnet, the solution containing released DNA-GOx conjugates then catalyzed the oxidation of neutral glucose into acidic gluconic acid and thus cause the pH change of the testing solution. Since the concentration Pb²⁺ in the sample, the amount of ssDNA dissociated, the amount of DNA-GOx conjugates released and the gluconic acid produced are dependent, the pH change detected by a portable meters was used to detect the concentration of Pb²⁺ in the sample quantitatively.

In 100 μL Buffer C, 2 μM DNA substrate (SEQ ID NO: 4) and 3 μM DNAzyme (SEQ ID NO: 3) was mixed with Pb²⁺ and stood at room temperature for 30 min. The reaction was quenched by mixing with 100 μL Buffer B. An aliquot of 150 μL of the resulting solution was transferred to the MBs residue prepared in Example 3 and mixed at room temperature for 30 min. After removal of the MBs by a magnet, 150 μL of the clear solution was mixed with 150 μL 0.8 M glucose in water. Finally, the solution was mixed with 500 μL water and tested by a portable pH meter after 30 minutes.

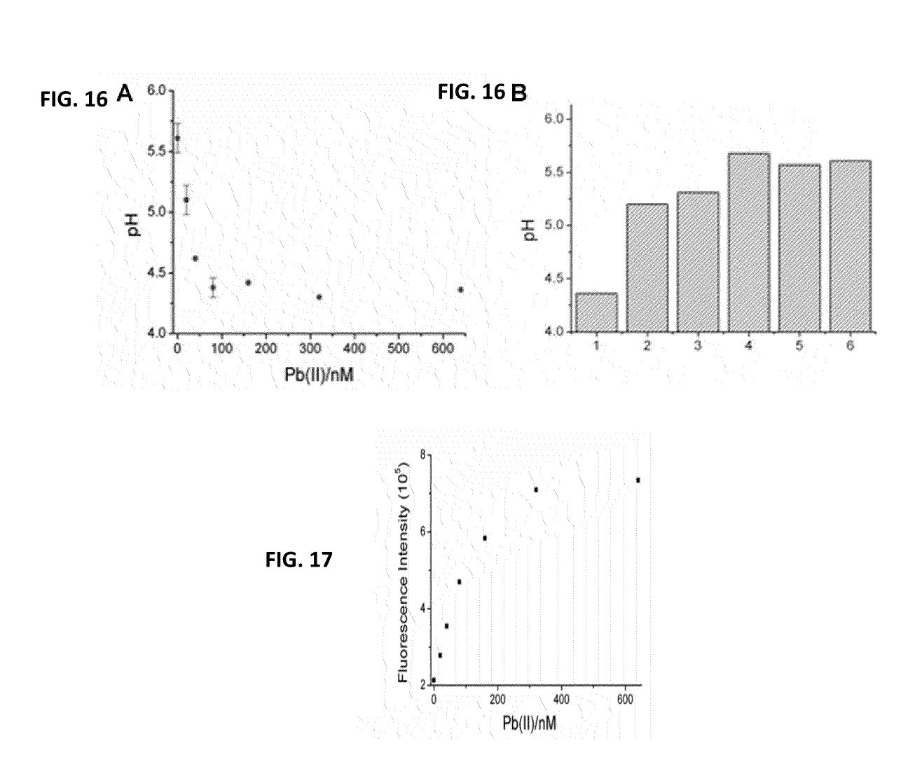

Using this approach, the detection of Pb²⁺ in water was successfully achieved. As shown in FIGS. 16A and 16B, the measured pH decreased with increasing amount of Pb²⁺ in the sample in the 0-100 nM range, and reached the plateau for samples with more Pb²⁺. A detection limit about 20 nM Pb²⁺ was obtained based on the definition of 3σ_{b}/slop, lower than the EPA regulated level of 72 nM in drinking water. The reason that the pH could hardly change further when Pb²⁺ in the samples exceeded 100 nM should be because of either all the cleavable DNA in FIG. 15A was cleaved so that the signal reached saturation or the plateau pH value was close to that of the oxidation product under the condition.

To confirm the immobilized DNA-GOx conjugates were actually released when mixed with samples containing Pb²⁺, fluorescein-labeled DNA-GOx conjugates were used under the same conditions. As illustrated in FIG. 17, increasing fluorescence was observed in the solution after removal of MBs, demonstrating that more DNA-GOx conjugates were released for the samples containing more Pb²⁺.

### Example 6

### Procedure for Cocaine Detection by pH Meter

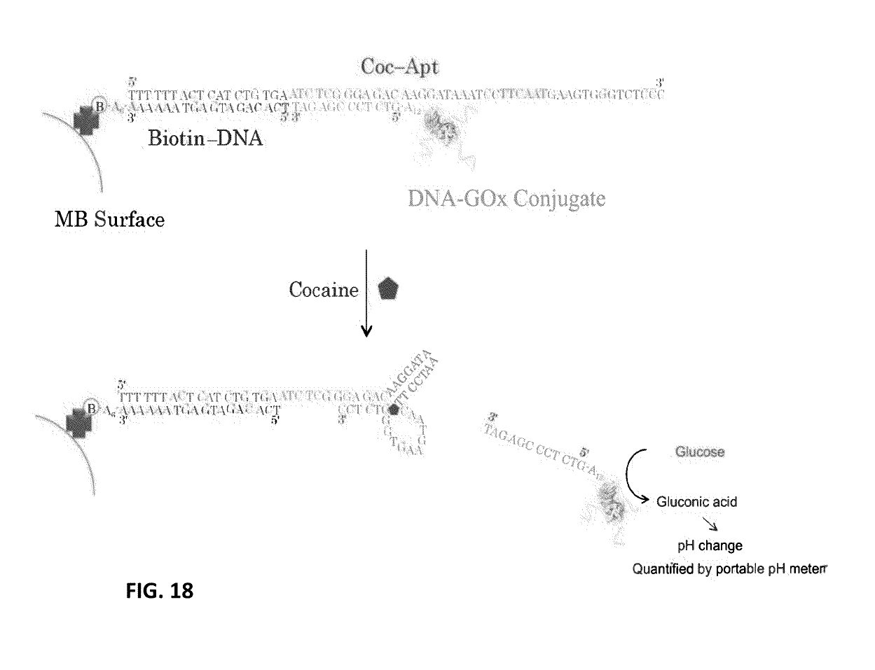

This example describes the use of a cocaine aptamer and DNA-GOx, both attached to magnetic beads, as generally illustrated in FIG. 18. One skilled in the art will appreciate that similar methods can be used to for other targets and aptamers.

FIG. 18 shows the principle of the method. First, DNA-GOx conjugates were immobilized on the surface of MBs through DNA hybridization of a cocaine aptamer^{11,12} (Coc-Apt). Upon the addition of sample solutions containing cocaine, cocaine bound to the aptamer and induced its structure switching, which disturbed the DNA hybridization between the aptamer and the DNA-GOx and caused the release of DNA-GOx from surface of MBs into solution. After removal of MBs by a magnet, the solution containing released DNA-GOx was mixed with glucose and the enzyme catalyze the oxidation reaction yielding gluconic acid. Finally, the pH of the testing buffer decreased as more gluconic acid was produced. One skilled in the art will appreciate that pH paper can be used instead of a meter, for example for qualitative or semi-quantitative detection.

An aliquot of 20 μL sample containing cocaine was transferred to the MBs residue prepared as above and mixed at room temperature for 40 min. After removal of the MBs by a magnet, 15 μL of the clear solution was mixed with 15 μL 0.5 M glucose in water. Finally, the solution was mixed with 570 μL water and tested by a portable pH meter after 30 minutes.

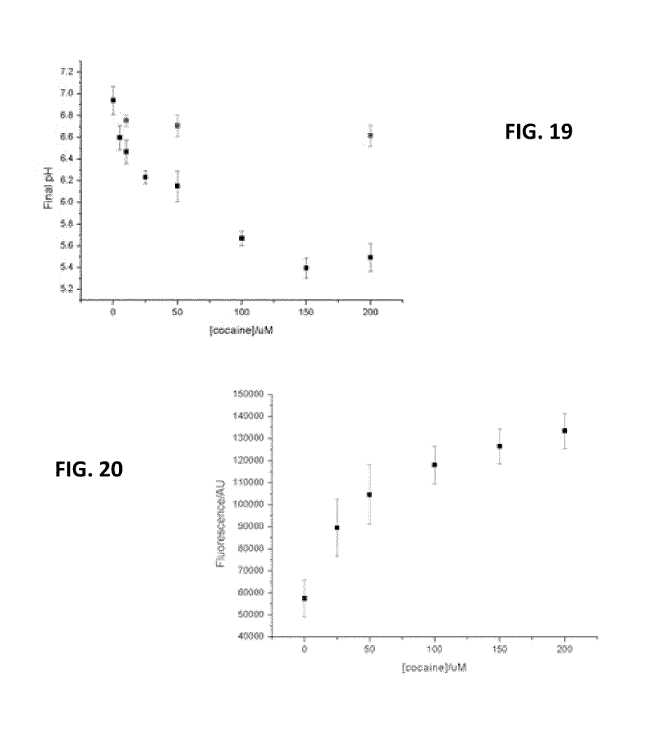

Applying this method to samples containing increasing amounts of cocaine, the results of pH measurements on the final solutions showed a decreasing trend (FIG. 19, black boxes). With cocaine in the samples up to 200 μM, the measured pH of the final solution decreased from around 6.9 to 5.4. A detection limit of around 10 μM cocaine was obtained based on the definition of 3σ_{b}/slop. In contrast, when samples containing adenosine were used as controls, only very mild pH change from 6.9 to 6.7 was observed (FIG. 8, red boxes on top).

Fluorescein-labeled DNA-GOx conjugates were used to confirm the DNA-GOx conjugates were released in the presence of cocaine. As shown in FIG. 20, increasing fluorescence was observed in the solution after removal of MBs for samples containing more cocaine, supporting the model shown in FIG. 18.

Thus, the disclosed methods can also detect organic molecules, such as cocaine, and demonstrates the generality of the method to a broad range of analytes that functional DNA can bind.

### Example 7

### Ricin Detection by pH Meter

This example describes the use of a DNA-GOx attached to graphene oxide, wherein the DNA is a ricin aptamer, as generally illustrated in FIG. 9. One skilled in the art will appreciate that similar methods can be used to for other targets and aptamers.

To demonstrate the general design of the assay, the method was used to detect a protein toxin, ricin. The biosensing platform is constructed according to the non-covalent assembly of aptamers on a graphene oxide (GO) surface which is induced by π-π stacking of DNA bases on GO. FIG. 9 shows the principle of the method. DNA-GOx conjugates were first immobilized on the surface of GO through π-π stacking. Upon the addition of ricin, the conformation of the aptamer on GO can be changed by complex formation induced by ricin. The weak binding between the complexes and GO surface can induce the release of DNA-GOx-ricin complex into solution. After separation, the released DNA-GOx-ricin complex catalyzes the production of gluconic acid from glucose, which decreases the solution pH. Since the concentration of ricin in the sample, the amount of DNA-GOx-ricin complex released and the gluconic acid produced are dependent, the pH change detected by a portable pH meters could then be used to detect the concentration of ricin in the sample quantitatively. One skilled in the art will appreciate that pH paper can be used instead of a meter, for example for qualitative or semi-quantitative detection.

50 μl of DNA-GOx-GO solution was mixed with 50 μL of different concentrations of ricin (from 0 to 2.5 μg/mL in buffer D) and stood at room temperature for 15 minutes. Subsequently, the solution was separated and 90 μL of the supernatant was transferred into 10 μL of 1 M glucose in Buffer D. After standing at room temperature for 30 minutes, 400 μL of water was added and the pH of the solution was measured by a portable pH meter. For detections in milk, the ricin stock solution in milk was diluted to different concentrations using buffer D (1:50 v/v), and the milk without ricin B chain were used as negative controls.

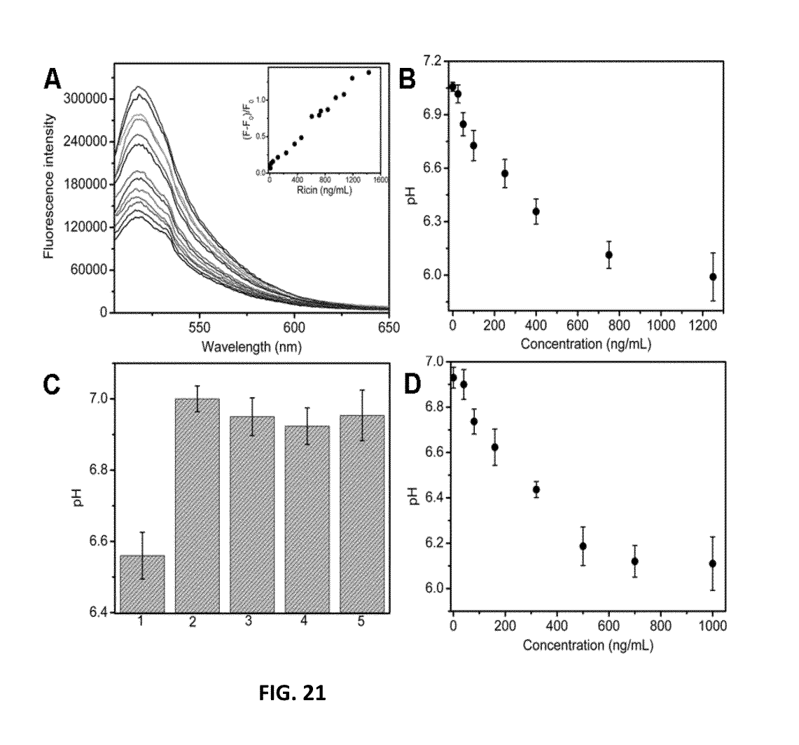

To demonstrate the feasibility of the method, fluorescence spectra was used to evaluate the release of FAM-labeled aptamer from the surface of GO by ricin. The fluorescence intensity decreased rapidly when GO was added into the FAM-aptamer solution of 50 nM, which was due to FRET between FAM and GO. Upon the addition of ricin, a significant fluorescence enhancement was observed, indicating that the competitive binding of the ricin with GO for FAM-labeled aptamer resulted in desorption of the FAM-labeled aptamer from the surface of GO (FIG. 21A). The fluorescence intensity increases with the increasing concentration of ricin. The plot of fluorescence intensity as a function of ricin concentration from 0 to 1.4 μg/mL is shown in the inset of FIG. 21A, supporting the hypothesis shown in FIG. 9.

Using this method, the detection of ricin in HEPES buffer was achieved. As shown in FIG. 21B, the measured pH decreased with increasing amount of ricin in the range of 0˜1.25 μg/mL, and reached the plateau for samples with more ricin. The detection limit was about 56 ng/mL according to the definition of 3σ_{b}/slope (σ_{b}, standard deviation of the blank samples). The sensitivity of this method is comparable to the previous reported colorimetric assay,¹³ SERS assay,¹⁴ and silicon photonic microring resonators assay.¹⁵ The sensor also exhibited excellent selectivity to ricin over other proteins, toxin and small molecules (FIG. 21C).

The ability of the method to detect ricin in complex liquid food matrices was also demonstrated. Commercial cow milk was used as the model of a matrix. The milk samples were spiked with ricin, diluted with HEPES buffer, filtered by 0.22 μm membrane to remove large casein micelles and fat globules, and analyzed using the competitive assay. FIG. 21D shows the dose-pH response curve for the ricin spiked into the cow milk. The detection limit was 107 ng/mL at 3σ. Considering the median lethal dose (LD₅₀) of ricin is around 22 μg/kg, the proposed assay for ricin in liquid foods is quite generous. This demonstrates that the methods provided herein can be used to detect targets in complex samples, such as a food sample.

### REFERENCES

(1) Daar, A. S.; Thorsteinsdottir, H.; Martin, D. K.; Smith, A. C.; Nast, S.; Singer, P. A. Nat. Genet. 2002, 32, 229-232.
(2) Mcbryde, W. A. E. Analyst 1969, 94, 337-&.
(3) Montagnana, M.; Caputo, M.; Giavarina, D.; Lippi, G. Clin. Chim. Acta 2009, 402, 7-13.
(4) Xiang, Y.; Wang, Z. D.; Xing, H.; Wong, N. Y.; Lu, Y. Anal. Chem. 2010, 82, 4122-4129.
(5) Xiang, Y.; Lu, Y. Anal. Chem. 2012, 84, 4174-4178.
(6) Xiang, Y.; Lu, Y. Nat. Chem. 2011, 3, 697-703.
(7) Liu, J. W.; Cao, Z. H.; Lu, Y. Chem. Rev. 2009, 109, 1948-1998.
(8) Li, J.; Lu, Y. J. Am. Chem. Soc. 2000, 122, 10466-10467.
(9) Liu, J. W.; Lu, Y. J. Am. Chem. Soc. 2003, 125, 6642-6643.
(10) Xiang, Y.; Tong, A.; Lu, Y. J. Am. Chem. Soc. 2009, 131, 15352-15357.
(11) Swensen, J. S.; Xiao, Y.; Ferguson, B. S.; Lubin, A. A.; Lai, R. Y.; Heeger, A. J.; Plaxco, K. W.; Soh, H. T. J. Am. Chem. Soc. 2009, 131, 4262-4266.
(12) Stojanovic, M. N.; de Prada, P.; Landry, D. W. J. Am. Chem. Soc. 2001, 123, 4928-4931.
(13) Schofield, C. L.; Mukhopadhyay, B.; Hardy, S. M.; McDonnell, M. B.; Field, R. A.; Russell, D. A. Analyst 2008, 133, 626
(14) He, L. L.; Lamont, E.; Veeregowda, B.; Sreevatsan, S.; Haynes, C. L.; Diez-Gonzalez, F.; Labuza, T. P. Chem. Sci. 2011, 2, 1579
(15) Shia, W. W.; Bailey, R. C. Anal. Chem. 2013, 85, 805

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

We claim:

1. A method for detecting a target, comprising
contacting a sample with (a) a recognition molecule specific for the target and (b) a solid support comprising glucose oxidase, under conditions sufficient to allow the target in the sample to bind to the recognition molecule and to release the glucose oxidase from the solid support;
separating the solid support from the released glucose oxidase;
contacting the released glucose oxidase with glucose, thereby generating gluconic acid; and
detecting a change in pH, wherein detection of a significant decrease in pH indicates the presence of the target agent in the sample, and an absence of detected significant decrease in pH indicates the absence of the target agent in the sample.

2. The method of claim 1, wherein the solid support comprises a bead, graphene oxide, a lateral flow device, or a microfluidic device.

3. The method of claim 1, further comprising quantifying the target, wherein the detected pH indicates an amount of target agent present.

4. The method of claim 1, wherein the gluconic acid is detected using a pH meter or pH paper.

5. The method of claim 1, wherein the target comprises a metal, microbe, cytokine, hormone, cell, nucleic acid molecule, spore, protein, recreational drug, or toxin.

6. The method of claim 1, wherein the solid support comprises
a first nucleic acid molecule having a 5′-end and a 3′-end, wherein the first nucleic acid is attached to the solid support by the 5′-end; and
a second nucleic acid molecule having a 5′-end and a 3′-end, wherein the 5′-end of the second nucleic acid molecule is hybridized to the 3′-end of the first nucleic acid molecule and wherein the 3′-end of the second nucleic acid molecule comprises the glucose oxidase.

7. The method of claim 6, wherein the recognition molecule comprises a DNAzyme specific for the target, and wherein the DNAzyme comprises an enzyme strand, a substrate strand, and optionally a RNA base in the substrate strand, wherein binding of the target to the DNAzyme cleaves the substrate strand at the RNA base into a 5′-end piece and a 3′-end piece, wherein the 5′-end piece of the substrate strand is complementary to the first nucleic acid molecule, and wherein the 5′-end piece of the substrate strand displaces the second nucleic acid molecule comprising the glucose oxidase from the first nucleic acid molecule, thereby releasing the glucose oxidase from the solid support.

8. The method of claim 7, wherein the solid support comprises a lateral flow device, and wherein:
contacting the sample with the DNAzyme specific for the target and the solid support comprising glucose oxidase comprises contacting the lateral flow device with the sample under conditions sufficient to allow the target in the sample to flow through the lateral flow device and bind to the DNAzyme on the lateral flow device, thereby forming a target-DNAzyme complex;
wherein separating the solid support from the released glucose oxidase comprises allowing the 5′-end piece of the substrate strand of the DNAzyme to flow to a region of the lateral flow device containing the attached first and second nucleic acid molecules, and wherein the 5′-end piece of the substrate strand displaces the second nucleic acid molecule comprising the glucose oxidase from the first nucleic acid molecule,
wherein contacting the released glucose oxidase with glucose comprises allowing the released second nucleic acid molecule comprising the glucose oxidase to flow to a region of the lateral flow device containing the glucose under conditions that permit the formation of gluconic acid.

9. The method of claim 1, wherein the solid support comprises
a first nucleic acid molecule having a 5′-end and a 3′-end, wherein the first nucleic acid is attached to the solid support by the 3′-end;
a second nucleic acid molecule having a 5′-end and a 3′-end, wherein the 3′-end of the second nucleic acid molecule is proximal to the 5′-end of the first nucleic acid molecule and wherein the 5′-end of the second nucleic acid molecule comprises the glucose oxidase; and
the recognition molecule specific for the target, wherein the recognition molecule comprises an aptamer nucleic acid molecule having a 5′-end and a 3′-end, wherein the aptamer nucleic acid molecule is complementary and hybridizes to the first nucleic acid molecule and to the second nucleic acid molecule, wherein the 3′-end of the aptamer nucleic acid molecule is not hybridized.

10. The method of claim 9, wherein binding of the target to the aptamer results in a conformational change in the 3′-end of the aptamer nucleic acid molecule and displaces the second nucleic acid molecule comprising the glucose oxidase from the aptamer nucleic acid molecule, thereby releasing the glucose oxidase from the solid support.

11. The method of claim 10, wherein the solid support comprises a lateral flow device, and wherein:
contacting the sample with the aptamer specific for the target and the solid support comprising glucose oxidase comprises contacting the lateral flow device with the sample under conditions sufficient to allow the target in the sample to flow through the lateral flow device and bind to the aptamer on the lateral flow device, thereby forming a target-aptamer complex, wherein the aptamer undergoes a conformational change;
wherein separating the solid support from the released glucose oxidase comprises allowing the released second nucleic acid molecule comprising the glucose oxidase to flow to a region of the lateral flow device containing the glucose under conditions that permit the formation of gluconic acid.

12. The method of claim 1, wherein the solid support comprises
the recognition molecule specific for the target, wherein the recognition molecule comprises an aptamer nucleic acid molecule having a first end and a second end, wherein the nucleic acid molecule is attached to the solid support by the first end and comprises the glucose oxidase on the second end.

13. The method of claim 12, wherein binding of the target to the aptamer results in a conformational change in the nucleic acid molecule and displaces the nucleic acid molecule comprising the glucose oxidase from the solid support, thereby releasing the glucose oxidase from the solid support.

14. The method of claim 12, wherein the solid support comprises a lateral flow device, and wherein:
contacting the sample with the aptamer specific for the target and the solid support comprising glucose oxidase comprises contacting the lateral flow device with the sample under conditions sufficient to allow the target in the sample to flow through the lateral flow device and bind to the aptamer on the lateral flow device, thereby forming a target-aptamer complex, wherein the aptamer undergoes a conformational change;
wherein separating the solid support from the released glucose oxidase comprises allowing the released nucleic acid molecule comprising the glucose oxidase to flow to a region of the lateral flow device containing the glucose under conditions that permit the formation of gluconic acid.

15. The method of claim 1, wherein:
the recognition molecule is bound to (a) the solid support and to (b) a target-glucose oxidase conjugate, under conditions sufficient to allow the target in the sample to compete with the target-glucose oxidase conjugate for binding to the recognition molecule on the solid support and to release the target-glucose oxidase conjugate from the solid support;
wherein separating the solid support from the released glucose oxidase comprises separating the solid support from unbound target and unbound target-glucose oxidase conjugate; and
wherein contacting the released glucose oxidase with glucose comprises contacting the unbound target-glucose oxidase conjugate with glucose, thereby generating gluconic acid.

16. The method of claim 15, wherein the recognition molecule is an antibody or a nucleic acid molecule.

17. The method of claim 1, wherein:
contacting a first recognition molecule specific for the target with a sample under conditions sufficient to allow the target in the sample to bind to the first recognition molecule, thereby creating a target-recognition molecule complex, wherein the recognition molecule is attached to a solid support;
contacting the target-recognition molecule complex with glucose oxidase, wherein the glucose oxidase is conjugated to a second recognition molecule specific for the target, thereby creating a target-recognition molecule-glucose oxidase recognition molecule complex;
contacting the glucose oxidase with glucose, thereby generating gluconic acid; and
detecting a change in pH, wherein detection of a significant decrease in pH indicates the presence of the target agent in the sample, and an absence of detected significant decrease in pH indicates the absence of the target agent in the sample.

18. The method of claim 17, wherein the first and the second recognition molecules are antibodies or nucleic acid molecules.

19. A sensor, comprising
(a) a solid support comprising
a first nucleic acid molecule having a 5′-end and a 3′-end, wherein the first nucleic acid is attached to the solid support by the 5′-end, and wherein the first nucleic acid is complementary to a 5′-end of a substrate strand of a DNAzyme specific for a target that can be detected by the sensor; and
a second nucleic acid molecule having a 5′-end and a 3′-end, wherein the 5′-end of the second nucleic acid molecule is hybridized to the 3′-end of the first nucleic acid molecule and wherein the 3′-end of the second nucleic acid molecule comprises glucose oxidase;
(b) a solid support comprising
a first nucleic acid molecule having a 5′-end and a 3′-end, wherein the first nucleic acid is attached to the solid support by the 3′-end;
a second nucleic acid molecule having a 5′-end and a 3′-end, wherein the 3′-end of the second nucleic acid molecule is proximal to the 5′-end of the first nucleic acid molecule and wherein the 5′-end of the second nucleic acid molecule comprises the glucose oxidase; and
an aptamer specific for a target that can be detected by the sensor, wherein the aptamer comprises a nucleic acid molecule having a 5′-end and a 3′-end, wherein the aptamer nucleic acid molecule is complementary and hybridizes to the first nucleic acid molecule and to the second nucleic acid molecule, wherein the 3′-end of the aptamer nucleic acid molecule is not hybridized
(c) a solid support comprising
an aptamer nucleic acid molecule having a first end and a second end, wherein the nucleic acid molecule is attached to the solid support by the first end and comprises glucose oxidase on the second end, and wherein the solid support comprises graphene oxide; or
(d) a solid support comprising
a recognition molecule bound to a target-glucose oxidase complex, wherein in the presence of the target in a sample the amount of target-glucose oxidase complex bound to the solid support decreases, and wherein the amount of target in the sample is proportional to the amount of unbound target-glucose oxidase complexes.

20. A lateral flow device or a microfluidic device comprising:
the sensor of claim 19.


SEQUENCE LISTING (14 sequences)

SEQ ID NO: 1 - DNA, 31 bp, Artificial Sequence
  Biotin-DNA for DNA-GOx immobilization onto magnetic beads for Pb2+ detection
aaaaaaaaaaaatatagtgtagcatcggaca

SEQ ID NO: 2 - DNA, 24 bp, Artificial Sequence
  DNA for DNA-GOx conjugation for Pb2+ detection
tgtccgatgctaaaaaaaaaaaaa

SEQ ID NO: 3 - DNA, 38 bp, Artificial Sequence
  Pb2+-dependent DNAzyme
acagacatctcttctccgagccggtcgaaatagtgtag

SEQ ID NO: 4 - DNA, 34 bp, Artificial Sequence
  Substrate for Pb2+-dependent DNAzyme
tgtccgatgctacactatnggaagagatgtctgt

SEQ ID NO: 5 - DNA, 24 bp, Artificial Sequence
  Biotin-DNA for DNA-GOx immobilization onto magnetic beads for cocaine detection
tcacagatgagtaaaaaaaaaaaa

SEQ ID NO: 6 - DNA, 24 bp, Artificial Sequence
  DNA for DNA-GOx conjugation for cocaine detection
aaaaaaaaaaaagtctcccgagat

SEQ ID NO: 7 - DNA, 52 bp, Artificial Sequence
  DNA for DNA-GOx conjugation for ricin detection
aaaaaaaaaaaaacacccaccgcaggcagacgcaacgcctcggagactagcc

SEQ ID NO: 8 - DNA, 62 bp, Artificial Sequence
  Cocaine aptamer
ttttttactcatctgtgaatctcgggagacaaggataaatccttcaatgaagtgggtctccc

SEQ ID NO: 9 - DNA, 57 bp, Artificial Sequence
  UO22+-dependent DNAzyme
aaaaccacgtccatctctgcagtcgggtagttaaaccgaccttcagacatagtgtag

SEQ ID NO: 10 - DNA, 41 bp, Artificial Sequence
  Substrate for UO22+-dependent DNAzyme
tgtccgatgctacactatraggaagagatggacgtggtttt

SEQ ID NO: 11 - DNA, 15 bp, Artificial Sequence
  biotin DNA for INF-gamma detection
tcacagatgagtagt

SEQ ID NO: 12 - DNA, 24 bp, Artificial Sequence
  DNA for DNA-GOX conjugation for IFN-gamma dectection
aaaaaaaaaaaaacaaccaaccca

SEQ ID NO: 13 - DNA, 50 bp, Artificial Sequence
  adenosine aptamer
ttttttactcatctgtgaagagaacctgggggagtattgcggaggaaggt

SEQ ID NO: 14 - DNA, 55 bp, Artificial Sequence
  IFN-gamma aptamer
tggggttggttgtgttgggtgttgtgtaaaaaaaaaaaaaactactcatctgtga